(19) United States
(12) Patent Application Publication
(10) Pub. No.: US 2023/0139443 A1
(43) Pub. Date: May 4, 2023

(54) TREATING AUTOSOMAL DOMINANT BESTROPHINOPATHIES AND METHODS FOR EVALUATING SAME

(71) Applicant: The Trustees of the University of Pennsylvania, Philadelphia, PA (US)

(72) Inventors: Karina E. Guziewicz, Broomall, PA (US); Artur V. Cideciyan, Lafayette Hill, PA (US); William A. Beltran, Wynnewood, PA (US); Samuel G. Jacobson, Penn Valley, PA (US); Gustavo D. Aguirre, Media, PA (US)

(21) Appl. No.: 17/904,899

(22) PCT Filed: Feb. 28, 2021
(86) PCT No.: PCT/US2021/020169

§ 371 Date: Aug. 24, 2022

Related U.S. Application Data
(60) Provisional application No. 62/983,046, filed on Feb. 28, 2020.
(60) Provisional application No. 62/983,052, filed on Feb. 28, 2020.

(51) Int. Cl. A61K 38/17; A61K 48/00; A61P 27/02; C12N 15/86; A61B 3/14; A61B 3/12

(52) U.S. Cl. CPC A61K 38/1709; A61K 48/0075; A61K 48/0058; A61P 27/02; C12N 15/86; A61B 3/14; A61B 3/12; C12N 2750/14143; C12N 2750/14145

(57) ABSTRACT

Methods for treating bestrophinopathies are provided herein. The method includes, administering to an eye of the subject a dose of a recombinant adeno-associated virus (rAAV) vector comprising a nucleic acid sequence encoding a human BEST1 protein, wherein the subject has at least one mutant BEST1 allele. Also provided are methods for evaluating treatments for retinal degeneration.

### STATEMENT OF GOVERNMENT SUPPORT

This invention was made with government support under EY006855 awarded by the National Institutes of Health. The government has certain rights in the invention.

### BACKGROUND OF THE INVENTION

Mutations in human BEST1 (hBEST1) result in a spectrum of retinal disease phenotypes collectively termed bestrophinopathies associated with pathognomonic macular lesions. To date, nearly 300 either monoallelic or biallelic mutations in hBEST1 have been identified and associated with inherited visual defects of variable onset, severity, and progression. The broad spectrum of clinical presentations in bestrophinopathies ranges from the widespread symptoms affecting peripheral retina and vitreous in a rare condition of vitreoretinochoroidopathy (ADVIRC) to the well-defined clinical abnormalities often limited to macula and paramacular areas in the central retina like in Best Vitelliform Macular Dystrophy (BVMD) and more extensive in autosomal recessive bestrophinopathy (ARB). BVMD, inherited as an autosomal dominant trait with incomplete penetrance, and the recessive form (ARB) are the most common and best explored juvenile macular dystrophies among bestrophinopathies, characterized by a markedly abnormal electrooculogram (EOG) accompanied by an excessive accumulation of lipofuscin material within cells of the retinal pigment epithelium (RPE), formation of focal and multifocal subretinal lesions, and consequently, loss of central vision.

While bestrophinopathies were first described in 1905, understanding of their pathological mechanism as well as any progress in the development of treatment has been hampered by the dearth of reliable animal models to carry out the mechanistic studies. Recent identification of spontaneous animal models of BEST1-associated retinopathies has proven crucial in the investigation of disease mechanisms and development of new therapeutic strategies. The spontaneous canine BEST1 disease model (cBEST; canine multifocal retinopathy, cmr) is a naturally occurring autosomal recessive disorder in dogs, which is caused by the same genetic defects as human bestrophinopathies, and captures the full range of clinical manifestations observed in patients. To date, cBest retinopathy has been identified in thirteen dog breeds and results from one of three distinct mutations in the canine BEST1 ortholog (cBEST1 -c.73C>T/p.R25*, -c.482G>A/p.G161D, or -c.1388delC/P463fs) inherited in an autosomal recessive fashion. All three mutations lead to a consistent clinical phenotype in homozygous affected dogs, and model all major aspects of the disease-associated mutations as well as their molecular consequences described in man. The spectrum of clinical and molecular features recapitulated, including the salient predilection of lesions in the canine macular region, makes cBest an extremely attractive model system not only for addressing principles behind the molecular pathology of bestrophinopathies, but also for validating new therapeutic strategies.

Improvements in methods for treating autosomal dominant diseases, that is, Best1-associated disorders caused by monoallelic Best1 gene mutations and for evaluating the effectiveness of potential treatments for bestrophinopathies is desired.

### SUMMARY OF THE INVENTION

In one aspect, a method of treating a bestrophinopathy in a subject is provided. The method includes administering to an eye of the subject a dose of a recombinant adeno-associated virus (rAAV) vector comprising a nucleic acid sequence encoding a human BEST1 protein. In one embodiment, the subject has one mutant BEST1 allele. In another embodiment, the bestrophinopathy is Best Vitelliform Macular Dystrophy (BVMD), Autosomal dominant vitreoretinochoroidopathy (ADVIRC), or Adult-onset vitelliform macular dystrophy (AVMD).

In another aspect, a method of evaluating a bestrophinopathy is provided. The method includes administering to an eye of the subject a dose of a recombinant adeno-associated virus (rAAV) vector comprising a nucleic acid sequence encoding a human BEST1 protein. In one embodiment, the subject has two mutant BEST1 alleles. In another embodiment, the subject has one mutant BEST1 allele. The method includes performing in vivo retinal cross-sectional imaging to evaluate one or more of a longitudinal reflectivity profile (LRP), assessment of IS/OS to retinal pigment epithelium (RPE) and/or ELM to RPE distance in light-adapted and/or dark-adapted eyes, and formation of light-potentiated subretinal microdetachments. In one embodiment, treatment efficacy is evaluated by one or more indicators of rescue of the retinal microarchitecture through restoration of RPE apical microvilli structure, a reestablishment of proper apposition between RPE cells and photoreceptor (PR) outer segments (cytoarchitecture of RPE-PR interface), and a restoration of the insoluble cone-specific interphotoreceptor matrix (IPM). In another embodiment, the retinal imaging is performed using an ultrahigh-resolution optical coherence tomography (OCT) to generate said LRP.

In another aspect, a method for evaluating a treatment for a bestrophinopathy is provided. The method includes obtaining a subject harboring a BEST1 gene mutation; administering a therapy; and measuring one or more indicators of rescue of the retinal microarchitecture, a restoration of RPE apical microvilli structure, a reestablishment of proper apposition between RPE cells and photoreceptor (PR) outer segments (cytoarchitecture of RPE-PR interface), and a restoration of the insoluble cone-specific interphotoreceptor matrix (IPM) to determine treatment efficacy.

In another aspect, a method of treating a bestrophinopathy in a subject is provided. The method includes administering to an eye of the subject a dose of a recombinant adeno-associated virus (rAAV) vector comprising a nucleic acid sequence encoding a human BEST1 protein, wherein the subject has at least one mutant BEST1 allele. In one embodiment, the dose of the rAAV vector is a) administered at a concentration of about 1.0×10¹⁰ vector genomes (vg)/ml to about 1.0×10¹³ vg/ml; or b) about 5.0×10⁸ vg per eye to about 5.0×10¹² vg per eye. In one embodiment, the subject is a canine, mouse, rat, non-human primate, or human.

In certain embodiments, the bestrophinopathy is Best Vitelliform Macular Dystrophy (BVMD), Autosomal dominant vitreoretinochoroidopathy (ADVIRC), Adult-onset vitelliform macular dystrophy (AVMD), retinitis pigmentosa (RP), or Microcornea, rod-cone dystrophy, or cataract. In another embodiment, rAAV vector is administered to the retina of the subject. In another embodiment, the rAAV vector is administered via subretinal, intravitreal, or suprachoroidal injection. In another embodiment, the nucleic acid sequence expresses the human BEST1 protein in the retinal pigment epithelium (RPE) of the eye. In another embodiment, the nucleic acid sequence encoding the BEST1 protein is under the control of a human VMD2 promoter (hVMD2). In yet another embodiment, the dose of the rAAV vector is administered at a concentration of about 1.0×10¹⁰ vg/ml to about 3.0×10¹² vg/ml, optionally about 1.5×10¹⁰ vg/ml. In yet another embodiment, the dose of rAAV vector is administered at a concentration of about 1.0×10¹¹ vg/ml to about 7.5×10¹¹ vg/ml. In still a further embodiment, the dose of rAAV vector is administered at a concentration of about 3.0×10¹¹ vg/ml, about 6.0×10¹¹ vg/ml, about 7.5×10¹¹ vg/ml to about 1.0×10¹³ vg/ml, or about 3.5×10¹² vg/ml. In another embodiment, the dose of rAAV vector is administered in a volume of between about 50 ul and 500 ul. In another embodiment, the dose of rAAV vector is administered in a volume of about 150 ul, about 300 ul, or about 500 ul. In yet another embodiment, the dose of rAAV vector administered is about 5.0×10⁸ vg per eye to about 1.5×10¹⁰ vg per eye, optionally about 7.5×10⁸ vg per eye.

In yet another embodiment, the dose of rAAV vector administered is about 1.0×10¹⁰ vg per eye to about 1.0×10¹¹ vg per eye, optionally, 4.5×10¹⁰ vg per eye. In yet another embodiment, the dose of rAAV vector administered is about 1.0×10¹¹ vg per eye to about 5.0×10¹² vg per eye. In still another embodiment, the dose of rAAV vector administered is about 1.0×10¹² vg per eye.

In another embodiment, the rAAV vector comprises an AAV2, AAV3, AAV4, AAV5, AAV6, AAV7, AAV8, AAV9, AAV10, AAV11, AAV12, LK01, LK02, LK03, AAV 4-1, AAV-2i8, Rh10, and/or Rh74 capsid, or a hybrid, chimera, or combination thereof. In one embodiment, the rAAV vector comprises an AAV2 capsid, or a hybrid, chimera, or combination thereof. In certain embodiments, the rAAV vector is an AAV2-hVMD2-hBEST1 vector.

In one embodiment, the dose of rAAV is administered to each eye of the subject. In another embodiment, the dose of rAAV is administered to one eye of the subject.

In a certain embodiment, the method does not further comprise administration of a nucleic acid composition that suppresses the expression or activity of the at least one mutant BEST1 allele.

In another embodiment, the treatment of the bestrophinopathy is evaluated. In certain embodiments, the evaluation includes performing in vivo retinal imaging to evaluate one or more of a longitudinal reflectivity profile (LRP), IS/OS to retinal pigment epithelium (RPE) distance in light-adapted and/or dark-adapted eyes, electrophysiology, dark-adapted kinetic perimetry and formation of light-potentiated subretinal microdetachments. Treatment efficacy is indicated by one or more of a rescue of retinal microarchitecture through restoration of RPE apical microvilli structure, and a reestablishment of proper apposition between RPE cells and photoreceptor (PR) outer segments (cytoarchitecture of RPE-PR interface).

In another embodiment, performing in vivo retinal imaging comprises one or more of fundus examination, cSLO/SD-OCT, measurement of rod outer segments, cone outer segments, ONL thickness, and ELM-RPE distance. In another embodiment, performing in vivo retinal imaging comprises evaluation for reactive gliosis and/or cell migration. In yet another embodiment, performing in vivo retinal imaging comprises evaluation for Muller glial trunks/projections penetrating ONL layer with astrogliosis.

In certain embodiments, the retinal imaging is performed using an ultrahigh-resolution optical coherence tomography (OCT) to generate said LRP.

In another embodiment, the method further includes comparing a measurement of a selected parameter to a measurement in a normal control, mutant disease control, pre-treatment control, earlier timepoint control, an untreated contralateral eye, or a retinal region outside of a treatment bleb.

In another embodiment, the method further includes obtaining a retina sample from the treated subject and a) labeling the sample with at least one RPE- and/or photoreceptor-specific marker; b) obtaining high-resolution confocal or wide-field fluorescence microscope with Differential Interference Contrast (DIC) option images of the RPE-PR interdigitation zone; and c) assessing one or more of length of RPE apical microvilli, structure of apical microvilli, ONL thickness, and structural integrity of IPM. In one embodiment, the marker is selected from BEST1, RPE65, EZRIN, pEZRIN, MCT1, CRALBP, F-actin, hCAR, an L-opsin, an M-opsin, an S-opsin, PNA, GFAP, Iba1, RDS/PRPH2, and RHO.

In another aspect, a method of identifying a subject in need of treatment for a bestrophinopathy is provided. The method includes performing in vivo retinal imaging on the subject to evaluate one or more of a longitudinal reflectivity profile (LRP), IS/OS to retinal pigment epithelium (RPE) distance in light-adapted and/or dark-adapted eyes, topological map, and formation of light-potentiated subretinal microdetachments; identifying retinal changes indicative of Best-1 disease selected from one or more of abnormal POS-RPE apposition and microarchitecture of RPE-PR interface, elongation of both ROS & COS associated with increased ELM-RPE distance, accumulation of subretinal debris at RPE apical surface, or within subretinal space; compromised IPM and defective ELM; fluctuation of ONL thickness associated with reactive gliosis and cell migration; schistic changes inner/outer retina; formation of subretinal & intraretinal scars; RPE monolayer hypertrophy, occasional severe deformation of individual RPE cells associated with ONL & INL thickness fluctuations; and Muller Glial trunks/projections penetrating ONL layer. A subject is identified as being in need of treatment for bestrophinopathy when one or more retinal changes indicative of Best1 disease is present.

In one embodiment, the in vivo retinal imaging comprises one or more of measurement of rod outer segments, cone outer segments, ONL thickness, and ELM-RPE distance. In another embodiment, the in vivo retinal imaging comprises evaluation for reactive gliosis and/or cell migration. In yet another embodiment, the in vivo retinal imaging comprises evaluation for Muller glial trunks/projections penetrating ONL layer with astrogliosis. In still another embodiment, the retinal imaging is performed using an ultrahigh-resolution optical coherence tomography (OCT) to generate said LRP. In another embodiment, the retinal imaging comprises cSLO/SD-OCT, electrophysiology, or adaptation kinetics.

In certain embodiments, the method further includes treating the subject when one or more retinal changes indicative of Best1 disease is present. In one embodiment, the subject is treated using a method as described herein.

Other aspects and advantages of the invention will be readily apparent from the following detailed description of the invention.

### BRIEF DESCRIPTION OF THE DRAWINGS

FIG. 1 shows confocal images illustrating the molecular pathology of cBest (R25*/R25*) mutant retina compared with to wild-type (WT) retinal tissue from control subject. Retinal cryosections were immunolabeled with anti-EZRIN (green) and human cone arrestin (red) combined with peanut agglutinin lectin (PNA, cyan) and DAPI (blue) to detail the structural alterations underlying loss of the native extracellular compartmentalization of cone photoreceptor outer segments and loss of interaction between RPE and the adjacent photoreceptor OS, resulting in subretinal microdetachment.

FIG. 2 shows a comparison of cross-sectional retina images of the retina infor WT, cBest-Heterozygous (R25*), and cBest-Homozygous (R25*/P463fs) models obtained using either the Spectralis SD/OCT or Leica/Bioptigen Envisu R2210 SD-OCTUHR systems. Longitudinal reflectivity profiles (LRP) based on these UHR images are also shown to the right (Leica/Bioptigen Envisu R2210) compared to magnified images from Spectralis SD-OCT (in the center (Spectralis) and right (Leica/Bioptigen Envisu R2210) column)s.

FIG. 3 shows results from ex vivo analyses of WT (top) and cBest heterozygous (R25*) (bottom) retinas in correlation to LRP images from UHR OCT and corresponding schematic drawings of retinal lamination.

FIG. 4 shows molecular pathology in cBest heterozygous (R25*) (top) and WT (bottom) retinas. Retinal cryosections from cbest-R25*-het and WT control retinasd were assayed with anti-EZRIN (green), hCAR (red) and PNA (white) to delineate RPE apical surface and associated microvilli, examine RPE-PR juction and IPM. Confocal micrographs were analyzed in comparison to generated LRP to determine the origin of LRP peaks and factors underlying the abnormal LRP in cBest-het mutant retina.

FIG. 5 shows a comparison of cross-sectional images from either the Spectralis SD-OCT or Leica/Bioptigen Envisu R2210 SD-UHR OCT system and corresponding immunolabeled sections for from WT, cBest heterozygous, and cBest homozygous mutant retinas.

FIG. 6 shows rescue of the retinal microarchitecture at the RPE/PR interface following administration of AAV-mediated BEST1 gene augmentation therapy.

FIGS. 7A-7D demonstrate the retinal phenotype of cBest1-heterozygous. and cBest1-homozygous dog models compared with wild type (WT). FIG. 7A shows ultra-high resolution fiber-based Fourier domain optical coherence tomography of wild type (WT) dog retina. The images show that the in vivo and ex vivo data correlate. FIG. 7B shows the retinal phenotype of cBest1-heterozygous (cBest-het) dog model. The abnormal microarchitecture of the RPE-PR interface in cBest-het mutant model is shown. Elongation of both ROS & COS associated with increased external limiting membrane (ELM)-RPE distance, presence of L/MS-& RDS (PRPH2)-positive debris at the RPE apical surface indicating abnormal POS-RPE apposition and interaction in cBest-hets. FIGS. 7C and 7D show a comparison of the 2-D (FIG. 7C) and 3-D (FIG. 7D) retinal imaging of wild type and cBest-het models. FIGS. 7C and 7D show significant lengthening of COS and ROS, as well as stretching and curving of the IS/OS.

FIGS. 8A and 8B demonstrate that activation of Muller glia (MG) cells and reactive astrogliosis promote inflammatory environment in cBest retina in both cBest-homozygous and cBest-heterozygous mutant models. Extension of Muller glia processes can be seen reaching RPE cells.

FIG. 8C demonstrates activation of Muller glia in cBest-het retina. 40X (top) and 100X (bottom) confocal images show reactive gliosis in cBest-hets. Upregulation of glial fibrillary acid protein (GFAP—in green) is an indicator of retinal stress. Also seen are fluctuation of ONL thickness (top panel), INL-ONL cell migration (top panel), and elevation of retinal surface (SS stretch—top panel).

FIG. 9 further demonstrates the retinal phenotype of cBest1-heterozygous dog model as compared to WT.

FIG. 10 demonstrates that AAV-mediated BEST1 gene augmentation therapy restores retinal homeostasis and prevents gliotic changes in cBest mutant retina post AAV-BEST1 injection. The activation of Muller glia is limited to untreated retinal region, which is associated with subretinal microdetachment.

FIG. 11 shows a summary of cBest-AR rAAV2-hBest1-injected eyes enrolled in the study. All eyes receiving a dosage of 1.15×10¹¹ or higher showed rescue.

FIG. 12 shows assessment of cBest-AR treated subjects up to 74 weeks post injection.

FIG. 13 shows cBest eyes dosing in comparison to published cBest subjects.

FIG. 14A-14D demonstrate RPE-photoreceptor interface structure in cBest mutant models and rescue of retinal microarchitecture post AAV-mediated BEST1 gene augmentation therapy (A) Canine WT control retina (age: 71 weeks), (B) cBest-R25*-heterozygous mutant (age: 16 weeks), (C) cBest-R25*/P463fs mutant-untreated retina (116 weeks), and (D) cBest-R25*/P463fs mutant retina AAV-BEST1-treated (Tx) examined at 74 weeks post subretinal injection. Structural abnormalities at the RPE-PR interface associated with expansion of subretinal space (ELM to RPE distance) and compromised interphotoreceptor matrix (IPM) detected in cBest mutant retina (B) cBest-het with monoallelic BEST1 mutation (arrow), and (C) cBest mutant harboring biallelic BEST1 mutation (bracket), assayed with PNA (peanut agglutinin lectin) marker (white). Note a remarkable restoration of the extracellular matrix in cBest AAV-BEST1 treated retina (D) comparable to the WT control (A). PNA: peanut agglutinin lectin known for its selective binding to the cone insoluble extracellular matrix microdomains of interphotoreceptor matrix (IPM). DAPI (4′,6-diamidino-2-phenylindole) a nuclear counterstain.

FIGS. 15A and 15B demonstrate reestablishment of lipid homeostasis post AAV-mediated BEST1 gene therapy in cBest (A) Spatial distribution of unesterified (free) cholesterol visualized by sterol-binding probe filipin (cobalt blue) in a normal and cBest1-R25*-mutant retina. Note the excess of autofluorescent RPE deposits in the diseased tissue. Histochemical detection of esterified cholesterol (cobalt blue) in a 12-month-old cBest vs age-matched control retina. Representative retinal cryosections from cBest and age-matched control stained with a fluorescent neutral lipids' tracer dye BODIPY 493/503 (green) along with quantification of EC-BODIPY 493/503 signals in POS layer between WT and cBest-R25* mutant retinas. The observed difference was assessed as statistically significant using unpaired t-test (*p<0.05). EC distribution profile in canine wild-type and cBest1-affected retinae assayed with a lysochrome Oil Red O (ORO, rose). ORO-positive inclusions within the affected RPE (arrows) and in the subretinal space are shown (close-up). Anti-4-HNE labeling (red) in the mutant vs control retina. A scattered distribution of HNE-adducts within outer segments was observed in cBest retina outlining the apical contour of the hypertrophic RPE cells). Nuclei were counterstained with propidium iodide or DAPI. (B) Restoration of subretinal space homeostasis in cBest-R25* mutant retina vs controls.

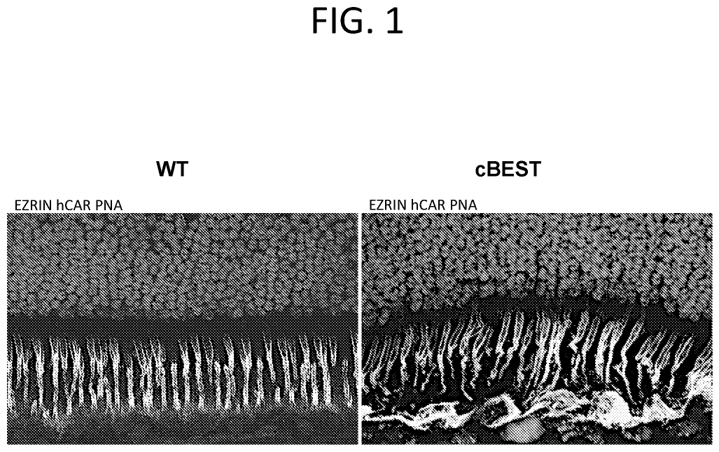

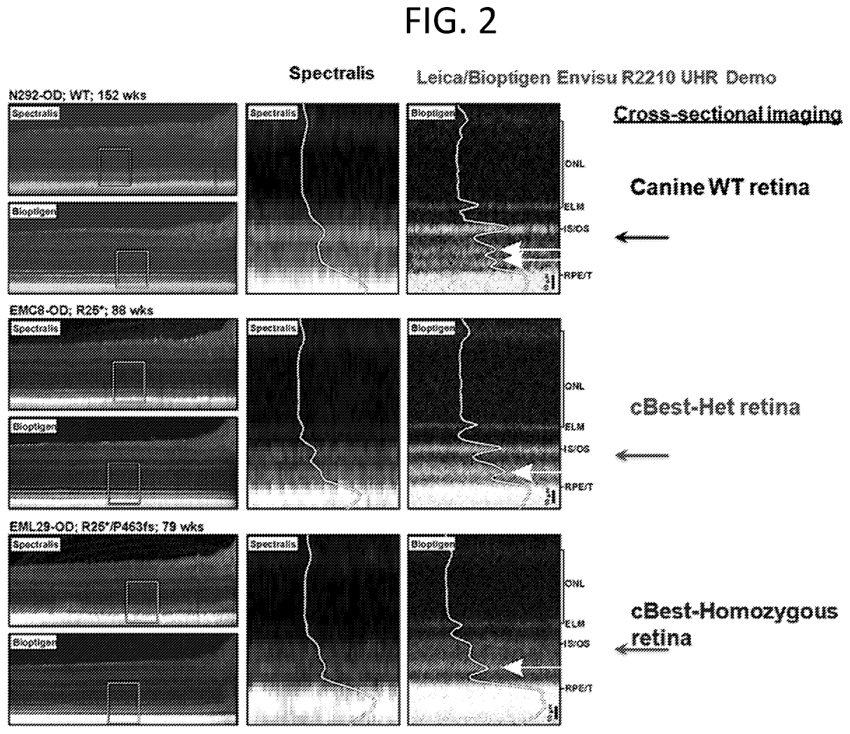

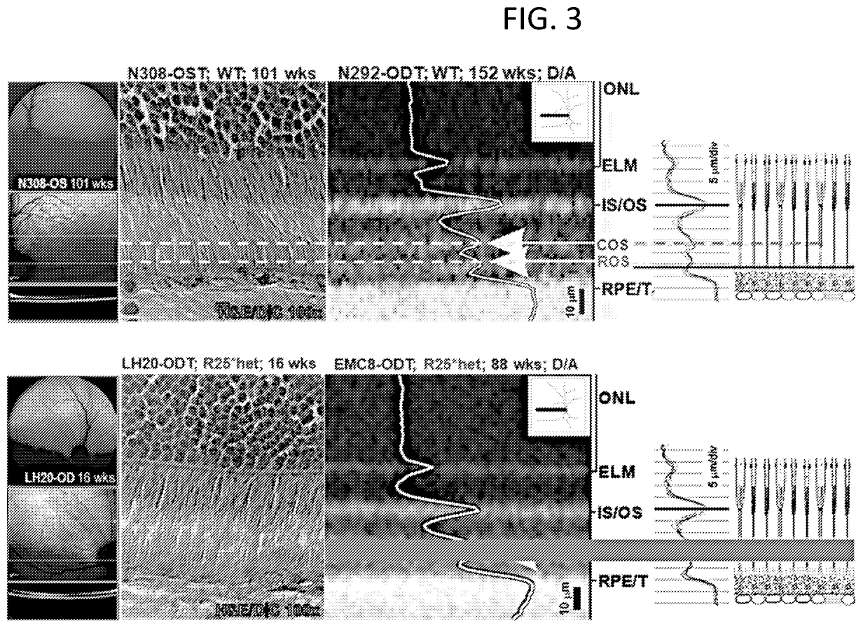

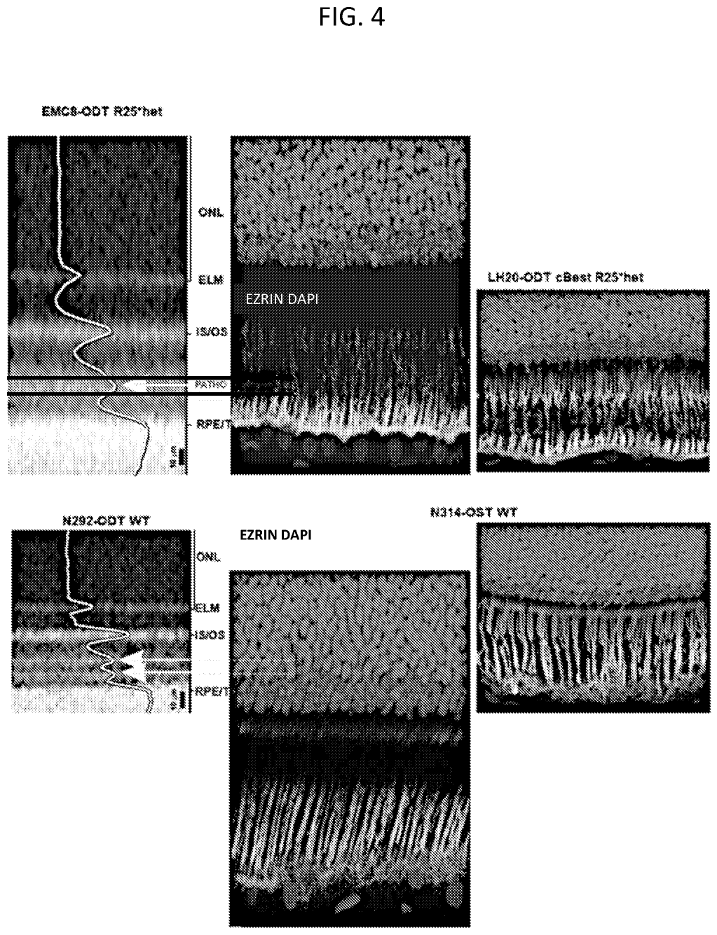

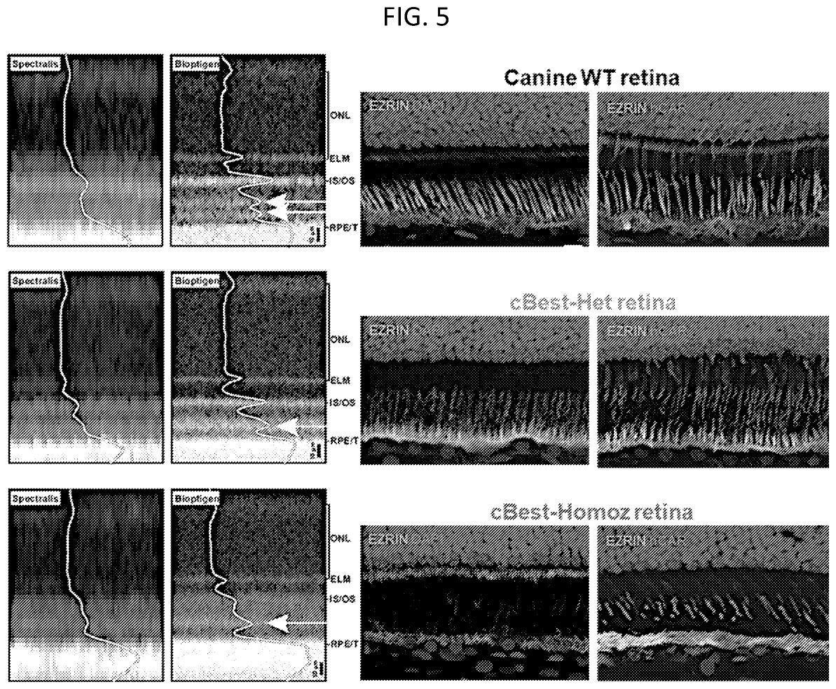

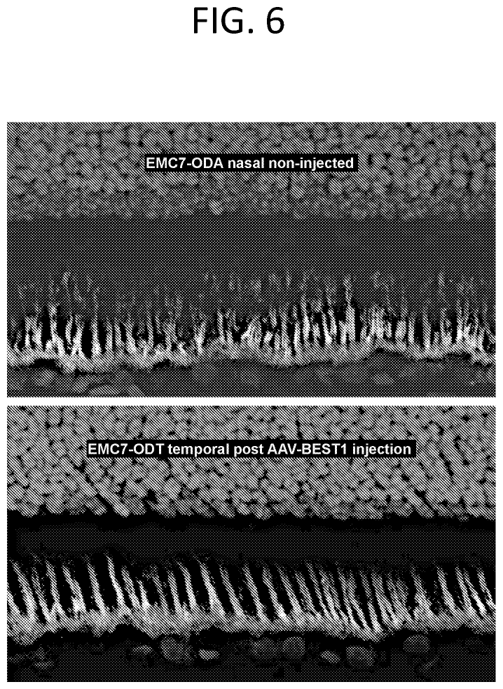

### DETAILED DESCRIPTION OF THE INVENTION

In certain aspects, provided herein are methods for treating bestrophinopathies. Also provided herein are methods for assessing retinal phenotype in subjects, including those harboring cBEST1 mutations. The methods are particularly suitable for evaluating the effectiveness of therapies in animal models used for research and development, as well as for diagnosing or assessing treatment of human subjects in a clinical setting. Accordingly, the subject being treated may be an animal model or a human subject having a mutation in a BEST1 allele.

In certain embodiments, provided herein are methods for treating, retarding, or halting progression of disease in a mammalian subject having an autosomal dominant (AD) BEST1-related ocular disease. In certain embodiments, the subject harbors a mutation in a BEST1 gene allele or has been identified as having or at risk of developing a bestrophinopathy, as described herein. The subject may be heterozygous for a specific mutation in the BEST1 gene, with one wild type allele, resulting in autosomal dominant (AD) bestrophinopathy. In certain embodiments, the AD bestrophinopathy may be Best vitelliform macular dystrophy (BVMD), adult-onset vitelliform macular dystrophy (AVMD), Vitreoretinochoroidopathy, Autosomal Dominant (ADVIRC), or retinitis pigmentosa (RP). In certain embodiments, the methods of treatment include providing a viral vector, as described herein.

A naturally occurring canine model of BEST1-associated retinopathies, canine Best (cBest), had been previously described. (Guziewicz et al, Bestrophin gene mutations cause canine multifocal retinopathy: a novel animal model for best disease. Invest Ophthalmol Vis Sci. 2007, incorporated herein by reference). Briefly, the model utilizes dogs that are homozygous mutant for the canine BEST1 (cBEST1) gene, and may result from any of three mutations identified at that locus. The homozygous mutant dogs of the model exhibit all major aspects of the human homozygous recessive BEST1 disease-associated mutations as well as their molecular consequences described in man.

As described herein, in vivo and ex vivo examination of cBEST1-heterozygous mutant (cBest-Het) dogs revealed an intermediate phenotype, indicating haploinsufficiency as a predominant mechanism underlying Best disease. As such, canine cBest-Het is the first spontaneous animal model for autosomal dominant Best vitelliform macular dystrophy (BVMD). The work described herein is the first identification of the cBest-Het phenotype, which enables use of the cBest-Het model for various diagnostic and therapeutic applications, as further described herein. The cBest-Het model may be useful in assessing potential efficacy of therapies, e.g., AAV mediated BEST1 gene augmentation therapies, for treatment of autosomal dominant BEST1-related ocular disorders such as BVMD. Moreover, the identification of phenotypical abnormalities in subjects harboring single copies of a mutant BEST1 allele potentially allows for improved methods of assessing therapies and evaluating treatment for bestrophinopathy in the human population, particularly in those with autosomal dominant disease. Furthermore, the observable and measurable features of the, at times, sub-clinical phenotype allow enhanced identification of individual subjects and patient populations that may be candidates for AAV mediated BEST1 gene augmentation therapies.

Also provided herein are compositions and methods for treating subjects having, or at risk of developing, autosomal dominant bestrophinopathy.

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein.

The term “about” as used herein when referring to a measurable value such as an amount, a temporal duration, and the like, is meant to encompass variations of up to ±10% from the specified value; as such variations are appropriate to perform the disclosed method.

As used in this specification and the appended claims, the singular forms “a,” “an,” and “the” include plural referents unless the content clearly dictates otherwise. As such, the terms “a” (or “an”), “one or more,” and “at least one” are used interchangeably herein. Thus, for example, reference to “a vector” includes two or more of the vectors, and the like.

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

BEST1 belongs to the bestrophin family of anion channels, which includes BEST2 (607335), BEST3 (607337), and BEST4 (607336). Bestrophins are transmembrane (TM) proteins that share a homology region containing a high content of aromatic residues, including an invariant arg-phe-pro (RFP) motif. The bestrophin genes share a conserved gene structure, with almost identical sizes of the 8 RFP-TM domain-encoding exons and highly conserved exon-intron boundaries.

The OMIM DB (www.ncbi.nlm.nih.gov/omim) lists 5 phenotypes associated with hBEST1 gene mutations, collectively termed ‘bestrophinopathies’, with the first affection described in 1905 (by Friedrich Best) and the latest one recognized in 2006 (Autosomal recessive bestrophinopathy (ARB)). The autosomal recessive form (ARB) can be caused by homozygous mutation (presence of the identical mutation on both alleles) or compound heterozygous mutation (both alleles of the same gene harbor mutations, but the mutations are different). As used herein, the term “biallelic” or “Autosomal Recessive (AR)” covers both causes.

Burgess et al., (Biallelic mutation of BEST1 causes a distinct retinopathy in humans. Am J Hum Genet. 2008 January; 82(1):19-31) described a distinct retinal disorder they designated autosomal recessive bestrophinopathy (ARB). Characteristics of the disorder included central visual loss, a characteristic retinopathy, an absent electrooculogram (EOG) light peak rise, and a reduced electroretinogram (ERG). None of the patients showed the vitelliform lesions characteristic of Best disease, but showed a diffuse irregularity of the reflex from the retinal pigment epithelium (RPE), including dispersed punctate flecks. All patients showed an accumulation of fluid within and/or beneath the neurosensory retina in the macula region. All patients were hyperopic, and 3 from 2 families also had angle-closure glaucoma. The severe reduction in the EOG light peak rise seen in all patients was similar to that seen both in Best disease and ADVIRC.

Autosomal dominant forms of bestrophinopathies are caused by monoallelic mutations in in the bestrophin gene (Bbestrophin-1). As used herein the term “Autosomal Dominant (AD) Best disease” may refer to any disease caused by a heterozygous mutation in the BEST1 gene. Such mutations may include a mutation in the heterozygous state. Such conditions include Best vitelliform macular dystrophy, Autosomal dominant vitreoretinochoroidopathy, Adult-onset vitelliform macular dystrophy, and MRCS syndrome.

Best vitelliform macular dystrophy (BVMD or VMD2), also called Best disease, is an early-onset autosomal dominant disorder characterized by large deposits of lipofuscin-like material in the subretinal space, which creates characteristic macular lesions resembling the yolk of an egg (‘vitelliform’). Although the diagnosis of Best disease is often made during the childhood years, it is more frequently made much later and into the sixth decade of life. In addition, the typical egg yolk-like lesion is present only during a limited period in the natural evolution of the disease; later, the affected area becomes deeply and irregularly pigmented and a process called ‘scrambling the egg’ occurs, at which point the lesion may appear as a ‘bull's eye.’ The disorder is progressive and loss of vision may occur. A defining characteristic of Best disease is a light peak/dark trough ratio of the electrooculogram (EOG) of less than 1.5, without aberrations in the clinical electroretinogram (ERG). Even otherwise asymptomatic carriers of BEST1 mutations, as assessed by pedigree, will exhibit an altered EOG. Histopathologically, the disease has been shown to manifest as a generalized retinal pigment epithelium (RPE) abnormality associated with excessive lipofuscin accumulation, regions of geographic RPE atrophy, and deposition of abnormal fibrillar material beneath the RPE, similar to drusen. Occasional breaks in the Bruch membrane with accompanying neovascularization have also been reported, although Best disease is not noted for extensive choroidal neovascularization.

BVMD often presents in several stages, although all individuals may not progress beyond the early stages.

Stage 1 (pre-vitelliform stage) consists of normal macula or subtle RPE pigment changes, EOG is abnormal and visual acuity (VA) is 20/20.

Stage 2 (vitelliform stage) consists of well-circumscribed, 0.5-5 mm round, elevated, yellow or orange lesion(s) bearing an egg-yolk appearance; usually centered on the fovea; may be multifocal; rest of the fundus has a normal appearance. VA is 20/20 to 20/50.

Stage 3 (pseudohypopyon stage) consists of yellow material which accumulate in the subretinal space in a cyst with a fluid level. The yellow material shifts with extended changes in position (60-90 min). This stage has been described in individuals aged 8-38 years. VA is 20/20 to 20/50.

Stage 4 (vitelliruptive stage) consists of scrambled egg appearance due to break up of the uniform vitelliform lesion. Pigment clumping and early atrophic changes may be noted. Visual acuity may deteriorate moderately. VA is 20/20 to 20/100.

Stage 5 (atrophic stage) consists of disappearance of the yellow material over time and an area of RPE atrophy remains. This appearance is difficult to distinguish from other causes of macular degeneration. Visual acuity can deteriorate more markedly at this stage. VA may reduce to less than 20/200.

Stage 6 (CNVM/cicatricial stage) occurs after the atrophic stage, where choroidal neovascularisation may develop and leading to a whitish subretinal fibrous scar. See, e.g., Maggon et al, Best's Vitelliform Macular Dystrophy, Med J Armed Forces India. 2008 October; 64(4): 379-381, which is incorporated herein by reference.

Adult-onset vitelliform macular dystrophy (AVMD) is one of the most common forms of macular degeneration. The age of AVMD onset is highly variable, but patients have a tendency to remain asymptomatic until the fifth decade. The clinical characteristics of AVMD are relatively benign, including a small subretinal vitelliform macular lesion, a slower progression of disease, and a slight deterioration in electrooculography (EOG). In some cases, AVMD is associated with autosomal dominant inheritance, with mutations in PRPH2, BEST1, IMPG1, or IMPG2.

Autosomal dominant vitreoretinochoroidopathy (ADVIRC or VRCP) is a disorder that affects several parts of the eyes, including the clear gel that fills the eye (the vitreous), the light-sensitive tissue that lines the back of the eye (the retina), and the network of blood vessels within the retina (the choroid). The eye abnormalities in ADVIRC can lead to varying degrees of vision impairment, from mild reduction to complete loss, although some people with the condition have normal vision. ADVIRC is caused by heterozygous mutation in the bestrophin-1 gene.

Retinitis pigmentosa is a retinal dystrophy belonging to the group of pigmentary retinopathies. Retinitis pigmentosa is characterized by retinal pigment deposits visible on fundus examination and primary loss of rod photoreceptor cells followed by secondary loss of cone photoreceptors. Patients typically have night vision blindness and loss of midperipheral visual field. As their condition progresses, they lose their far peripheral visual field and eventually central vision as well. Retinitis pigmentosa-50 (RP50) is caused by heterozygous mutation in the BEST1 gene, while certain types of retinitis pigmentosa can be autosomal recessive.

MRCS syndrome (Microcornea, rod-cone dystrophy, cataract, and posterior staphyloma) is a rare, genetic retinal dystrophy disorder characterized by bilateral microcornea, rod-cone dystrophy, cataracts and posterior staphyloma, in the absence of other systemic features. Night blindness is typically the presenting manifestation and nystagmus, strabismus, astigmatism and angle closure glaucoma may be associated findings. Progressive visual acuity deterioration, due to pulverulent-like cataracts, results in poor vision ranging from no light perception to 20/400. MRCS is caused by heterozygous mutation in the BEST1 gene.

In certain embodiments, provided herein are methods for treating, retarding, or halting progression of blindness in a mammalian subject having an autosomal dominant BEST1-related ocular disease. In certain embodiments, the subject harbors a mutation in a BEST1 gene allele or has been identified as having or at risk of developing a bestrophinopathy, as described herein. The subject may be heterozygous for a specific mutation in the BEST1 gene, with one wild type allele. In certain embodiments, the subject is heterozygous for a mutant BEST1 allele resulting in autosomal dominant bestrophinopathy. The AD bestrophinoapthy may be selected from BVMD, AVMD, ADVIRC, RP and MRCS. In certain embodiments, the methods of treatment include providing a viral vector, as described herein.

In certain embodiments of this invention, the subject has an “ocular disease,” e.g., an autosomal dominant BEST1-related ocular disease. Clinical signs of such ocular diseases include, but are not limited to, decreased peripheral vision, retinal degeneration, decreased central (reading) vision, decreased night vision, loss of color perception, reduction in visual acuity, decreased photoreceptor function, pigmentary changes, and ultimately blindness.

Retinal degeneration is a retinopathy which consists in the deterioration of the retina caused by the progressive death of its cells. There are several reasons for retinal degeneration, including artery or vein occlusion, diabetic retinopathy, R.L.F./R.O.P. (retrolental fibroplasia/retinopathy of prematurity), or disease (usually hereditary). Signs and symptoms of retinal degeneration include, without limitation, impaired vision, night blindness, retinal detachment, light sensitivity, tunnel vision, and loss of peripheral vision to total loss of vision. Retinal degeneration and remodeling encompass a group of pathologies at the molecular, cellular and tissue levels that are initiated by inherited retinal diseases such as those described herein and other insults to the eye/retina including trauma and retinal detachment. These retinal changes and apparent plasticity result in neuronal rewiring and reprogramming events that include alterations in gene expression, de novo neuritogenesis as well as formation of novel synapses, creating corruptive circuitry in bipolar cells through alterations in the dendritic tree and supernumerary axonal growth. In addition, neuronal migration occurs throughout the vertical axis of the retina along Müller cell columns showing altered metabolic signals, and retinal pigment epithelium (RPE) invades the retina forming the pigmented bone spicules that have been classic clinical observations of RP diseases. See, retinal degeneration, remodeling and plasticity by Bryan William Jones, Robert E. Marc and Rebecca L. Pfeiffer.

As used herein, the term “subject” means a mammalian animal, including a human, a veterinary or farm animal, a domestic animal or pet, and animals normally used for research. In certain embodiments, the subject of these methods is a human. In certain embodiments, the subject is a canine. In yet other embodiments, the subject is a non-human primate. Still other suitable subjects include, without limitation, murine, rat, feline, porcine, bovine, ovine, and others. As used herein, the term “subject” is used interchangeably with “patient.” In certain embodiments, the subject is a laboratory animal suitable for research purposes (including, but not limited to, mouse, rat, canine, and non-human primate) that has been genetically modified, for example, to introduce a mutation in an endogenous BEST1 gene or to introduce a transgene encoding a mutant BEST1. In certain embodiments, the animal subject has been modified to express a heterologous BEST1 gene, such as hBEST1 or a mutant hBEST1. In another embodiment, the animal subject is a cBEST1-heterozygous mutant. In certain embodiments, the subject is a cBest-heterozygous mutant model dog, as described herein. Transgenic animals can be generated produced by any method known to those of ordinary skill in the art (for example, a zinc finger nuclease, a TALEN and/or a CRISPR/Cas nuclease system).

In certain embodiments, the subject is a human at risk of developing bestrophinopathy (e.g., has a family history of bestrophinopathy) or has one or more confirmed BEST1 gene mutations. In yet another embodiment, the subject has shown clinical signs of a bestrophinopathy. In yet a further embodiment, the subject has shown signs of retinopathy that are also indicative of bestrophinopathy. In certain embodiments, the subject has been diagnosed with a bestrophinopathy. In yet another embodiment, the subject has not yet shown clinical signs of a bestrophinopathy. In one embodiment, the subject has, or is at risk of developing, an AD bestrophinopathy. In one embodiment, the bestrophinopathy is BVMD. In another embodiment, the bestrophinopathy is AVMD. In another embodiment, the bestrophinopathy is ADVIRC. In another embodiment, the bestrophinopathy is RP. In another embodiment, the bestrophinopathy is MRCS.

Although the diagnosis of Best disease is often made during the childhood years, it is more frequently made much later and into the sixth decade of life, using traditional techniques such as fundus examination and electrooculogram (EOG). The subtle phenotypic changes identified herein are useful in diagnosing AD Best disease earlier, and in individuals lacking the gross retinal and visual changes previously used for identification. Thus, in certain embodiments, the techniques described herein are used to identify a subject as having, or at risk of developing, autosomal dominant Best disease. In other embodiments, the techniques described here are used to identify a subject for suitability to receive gene replacement therapy for Best disease, such as the AAV mediated BEST1 gene augmentation therapies described herein.

In one embodiment, the subject is 10 years of age or less. In another embodiment, the subject is 15 years of age or less. In another embodiment, the subject is 20 years of age or less. In another embodiment, the subject is 25 years of age or less. In another embodiment, the subject is 30 years of age or less. In another embodiment, the subject is 35 years of age or less. In another embodiment, the subject is 40 years of age or less. In another embodiment, the subject is 45 years of age or less. In another embodiment, the subject is 50 years of age or less. In another embodiment, the subject is 55 years of age or less. In another embodiment, the subject is 60 years of age or less. In another embodiment, the subject is 65 years of age or less. In another embodiment, the subject is 70 years of age or less. In another embodiment, the subject is 75 years of age or less. In another embodiment, the subject is 80 years of age or less. In another embodiment, the subject is a neonate, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 years of age or greater.

As used herein, the term “treatment,” and variations thereof such as “treat” or “treating,” refer to clinical intervention in an attempt to alter the natural course of the individual being treated and can be performed either for prophylaxis or during the course of clinical pathology. “Treatment” can thus include one or more of reducing onset or progression of an ocular disease (such as bestrophinopathy), preventing disease, reducing the severity of the disease symptoms, or retarding their progression, including the progression of blindness, removing the disease symptoms, delaying onset of disease or monitoring progression of disease or efficacy of therapy in a given subject.

Thus, in certain embodiment, a therapy is administered before disease onset. In another embodiment, a therapy is administered prior to the initiation of vision impairment or loss. In another embodiment, a therapy is administered after initiation of vision impairment or loss. In yet another embodiment, a therapy is administered when less than 90% of the rod and/or cones or photoreceptors are functioning or remaining, as compared to a non-diseased eye.

In yet another embodiment, a therapy is administered when the subject being treated exhibits symptoms of stage I (the pre-vitelliform stage) to stage III (the vitelliruptive stage or the pseudo-hypopyon stage) of BVMD. In another embodiment, therapy is administered prior to exhibiting the symptoms of stage I. In another embodiment, therapy is administered after exhibiting the symptoms of stage I. In another embodiment, therapy is administered prior to exhibiting the symptoms of stage II. In another embodiment, therapy is administered after exhibiting the symptoms of stage II. In another embodiment, therapy is administered prior to exhibiting the symptoms of stage III. In another embodiment, therapy is administered after exhibiting the symptoms of stage III. In another embodiment, therapy is administered prior to exhibiting the symptoms of stage IV. In another embodiment, therapy is administered after exhibiting the symptoms of stage IV. In another embodiment, therapy is administered prior to exhibiting the symptoms of stage V. In another embodiment, therapy is administered after exhibiting the symptoms of stage V.

As used herein, “therapy” refers to any form of intervention intended to treat an existing disease condition in a subject or reduce, delay, inhibit or eliminate the onset or progression of disease or symptoms of disease in a subject. A therapy may be a gene augmentation therapy intended to supplement, restore, or enhance expression levels of a gene by providing a nucleic acid encoding a functional protein. Thus, in certain embodiments, the methods include administering a vector, in particular a gene therapy vector. In certain embodiments, the therapy is a recombinant AAV with a canine BEST1 (cBEST1) or human BEST1 (hBEST1). Suitable vectors may also encode components of a genome editing system (e.g., CRISPR/Cas) designed to, for example, insert a gene sequence, replace a gene sequence or part of a gene sequence, or correct a mutation in an endogenous BEST1 gene sequence.

The term “heterologous” as used to describe a nucleic acid sequence or protein means that the nucleic acid or protein was derived from a different organism or a different species of the same organism than the host cell or subject in which it is expressed.

The term “transgene” as used herein means an exogenous or engineered protein-encoding nucleic acid sequence that is under the control of a promoter or expression control sequence in an expression cassette, rAAV genome, recombinant plasmid or production plasmid, vector, or host cell described in this specification. In certain embodiments, the transgene is a BEST1 sequence, encoding a functional BEST1 protein, or a fragment thereof.

In certain embodiments, the methods include administering a viral vector to a subject. Suitable viral vectors are preferably replication defective and selected from amongst those which target ocular cells. Viral vectors may include any virus suitable for gene therapy wherein a vector includes a nucleic acid sequence encoding for protein intended mediate a therapeutic effect in the subject. Suitable gene therapy vectors include, but are not limited to adenovirus; herpes virus; lentivirus; retrovirus; parvovirus, etc. However, for ease of understanding, the adeno-associated virus is referenced herein as an exemplary viral vector.

Thus, in one aspect, a recombinant adeno-associated virus (rAAV) vector is provided. The rAAV compromises an AAV capsid, and a vector genome packaged therein. The vector genome comprises, in one embodiment: (a) an AAV 5′ inverted terminal repeat (ITR) sequence; (b) a promoter; (c) an optional enhancer; (d) a coding sequence encoding a human BEST1; (e) a polyA tail; and (f) an AAV 3′ ITR. In one embodiment, the BEST1 sequence encodes a full length bestrophin protein. In one embodiment, the BEST1 sequence is the protein sequence of Uniprot Accession No. 076090-1, which is incorporated herein by reference. (See, e.g., Guziewicz et al, PNAS. 2018 Mar. 20; 115 (12):E2839-E2848, which is incorporated by reference herein).

In certain embodiments, the methods include delivery of a vector (e.g. a gene therapy vector) having a nucleic acid sequence encoding a normal BEST1 gene, or fragment thereof. The term “BEST1” as used herein, refers to the full-length gene itself or a functional fragment, as further defined below. The nucleic acid sequence encoding a normal BEST1 gene, or fragment thereof, may be derived from any mammal which natively expresses the BEST1 gene, or homolog thereof. In certain embodiments, the BEST1 gene sequence is derived from the same mammal that the subject is intended to treat. Thus, in certain embodiments, the BEST1 gene is derived from a human sequence (as provided, for example, in any of NM_001139443.1, NM_001300786.1, NM_001300787.1, NM_001363591.1 NM_001363592.1 NM_001363593.1, and NM_004183.4). In certain embodiments, the BEST1 sequence encodes a protein having an amino acid sequence of UniProtKB—O76090-1, O76090-3, or O76090-4. In yet other embodiments, the BEST1 gene is derived from a canine sequence (as provided, for example, in NM_001097545.1). In certain embodiments, the BEST1 sequence encodes a protein having the amino acid sequence of UniProtKB-A5H7G8-1. In certain embodiments of the methods a human BEST1 (hBEST1) gene is delivered to a mammal other than a human (such as a canine, rat, mouse, or non-human primate model) to, for example, evaluate the efficacy of a therapy. In certain embodiment, the BEST1 sequence is the sequence of the full length human BEST1. By the term “fragment” or “functional fragment”, it is meant any fragment that retains the function of the full-length protein, although not necessarily at the same level of expression or activity. Functional fragments of human, or other BEST1 sequences may be determined by one of skill in the art. In certain embodiments, the BEST1 sequence is derived from a canine. In other embodiments, certain modifications are made to the BEST1 sequence in order to enhance the expression in the target cell. Such modifications include codon optimization, (see, e.g., U.S. Pat. Nos. 7,561,972; 7,561,973; and 7,888,112, incorporated herein by reference).

The term “adeno-associated virus,” “AAV,” or “AAV serotype” as used herein refers to the dozens of naturally occurring and available adeno-associated viruses, as well as artificial AAVs. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, AAV1, AAV2, AAV3, AAV4, AAV5, AAV6, AAV7, AAV8, AAV9, AAV8 bp, AAV2-7m8 and AAVAnc80, variants of any of the known or mentioned AAVs or AAVs yet to be discovered or variants or mixtures thereof. See, e.g., WO 2005/033321, which is incorporated herein by reference. In another embodiment, the AAV is selected from AAV10, AAV11, AAV12, LK01, LK02, LK03, AAV 4-1, AAV-2i8, Rh10, and/or Rh74. In another embodiment, the AAV capsid is an AAV8 bp capsid, which preferentially targets bipolar cells. See, WO 2014/024282, which is incorporated herein by reference. In another embodiment, the AAV capsid is an AAV2-7m8 capsid, which has shown preferential delivery to the outer retina. See, Dalkara et al, In Vivo-Directed Evolution of a New Adeno-Associated Virus for Therapeutic Outer Retinal Gene Delivery from the Vitreous, Sci Transl Med 5, 189ra76 (2013), which is incorporated herein by reference. In one embodiment, the AAV capsid is an AAV8 capsid. In another embodiment, the AAV capsid an AAV9 capsid. In another embodiment, the AAV capsid an AAV5 capsid. In another embodiment, the AAV capsid an AAV2 capsid.

As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized AAV capsid. Pseudotyped vectors, wherein the capsid of one AAV is replaced with a heterologous capsid protein, are useful in the invention. In one embodiment, AAV2/5 and AAV2-7m8 are exemplary pseudotyped vectors.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

For packaging an expression cassette or rAAV genome or production plasmid into virions, the ITRs are the only AAV components required in cis in the same construct as the expression cassette. In one embodiment, the coding sequences for the replication (rep) and/or capsid (cap) are removed from the AAV genome and supplied in trans or by a packaging cell line in order to generate the AAV vector.

Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated, even if subsequently reintroduced into the natural system. Such polynucleotides could be part of a vector and/or such polynucleotides or polypeptides could be part of a composition, and still be isolated in that such vector or composition is not part of its natural environment.

In yet another system, the expression cassette flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety.

The methods used to construct any embodiment of this invention are known to those with skill in nucleic acid manipulation and include genetic engineering, recombinant engineering, and synthetic techniques. See, e.g., Green and Sambrook et al, Molecular Cloning: A Laboratory Manual, Cold Spring Harbor Press, Cold Spring Harbor, N.Y. (2012). Similarly, methods of generating rAAV virions are well known and the selection of a suitable method is not a limitation on the present invention. See, e.g., K. Fisher et al, (1993) J. Virol., 70:520-532 and U.S. Pat. No. 5,478,745.

In certain embodiments, the rAAV expression cassette, the vector, and/or the virus comprises AAV inverted terminal repeat sequences, a nucleic acid sequence that encodes BEST1, and expression control sequences that direct expression of the encoded proteins in a host cell. In other embodiments, the rAAV expression cassette, the virus, and/or the vector further comprises one or more of an intron, a Kozak sequence, a polyA, post-transcriptional regulatory elements and others. In one embodiment, the post-transcriptional regulatory element is Woodchuck Hepatitis Virus (WHP) Posttranscriptional Regulatory Element (WPRE).

The expression cassettes, vectors and plasmids include other components that can be optimized for a specific species using techniques known in the art including, e.g., codon optimization, as described herein.

The components of the cassettes, vectors, plasmids and viruses or other compositions described herein include a promoter sequence as part of the expression control sequences. In one embodiment, the promoter is the native hVMD2 promoter. In another embodiment, the promoter is cell-specific. The term “cell-specific” means that the particular promoter selected for the recombinant vector can direct expression of the BEST1 coding sequence in a particular ocular cell type. In one embodiment, the promoter is specific for expression of the transgene in RPE. In one embodiment, the promoter is specific for expression of the transgene in photoreceptor cells. In another embodiment, the promoter is specific for expression in the rods and cones. In another embodiment, the promoter is specific for expression in the rods. In another embodiment, the promoter is specific for expression in the cones. In one embodiment, the photoreceptor-specific promoter is a human rhodopsin kinase promoter. The rhodopsin kinase promoter has been shown to be active in both rods and cones. See, e.g., Sun et al, Gene Therapy with a Promoter Targeting Both Rods and Cones Rescues Retinal Degeneration Caused by AIPL1 Mutations, Gene Ther. 2010 January; 17(1): 117-131, which is incorporated herein by reference in its entirety. In one embodiment, the promoter is modified to add one or more restriction sites to facilitate cloning.

In one embodiment, the promoter is the native hVMD2 promoter or a modified version thereof. See, Guziewicz et al., PLoS One. 2013 Oct. 15; 8 (10):e75666, which is incorporated herein by reference.

In one embodiment, the promoter is a human rhodopsin promoter. In one embodiment, the promoter is modified to include restriction on the ends for cloning. See, e.g., Nathans and Hogness, Isolation and nucleotide sequence of the gene encoding human rhodopsin, PNAS, 81:4851-5 (August 1984), which is incorporated herein by reference in its entirety. In another embodiment, the promoter is a portion or fragment of the human rhodopsin promoter. In another embodiment, the promoter is a variant of the human rhodopsin promoter.

Other exemplary promoters include the human G-protein-coupled receptor protein kinase 1 (GRK1) promoter (Genbank Accession number AY327580). In another embodiment, the promoter is a 292 nt fragment (positions 1793-2087) of the GRK1 promoter (See, Beltran et al, Gene Therapy 2010 17:1162-74, which is hereby incorporated by reference in its entirety). In another preferred embodiment, the promoter is the human interphotoreceptor retinoid-binding protein proximal (IRBP) promoter. In one embodiment, the promoter is a 235 nt fragment of the hIRBP promoter. In one embodiment, the promoter is the RPGR proximal promoter (Shu et al, IOVS, May 2102, which is incorporated by reference in its entirety). Other promoters useful in the invention include, without limitation, the rod opsin promoter, the red-green opsin promoter, the blue opsin promoter, the cGMP-p-phosphodiesterase promoter (Qgueta et al, IOVS, Invest Ophthalmol Vis Sci. 2000 December; 41(13):4059-63), the mouse opsin promoter (Beltran et al 2010 cited above), the rhodopsin promoter (Mussolino et al, Gene Ther, Jul. 2011, 18(7):637-45); the alpha-subunit of cone transducin (Morrissey et al, BMC Dev, Biol, Jan. 2011, 11:3); beta phosphodiesterase (PDE) promoter; the retinitis pigmentosa (RP1) promoter (Nicord et al, J. Gene Med, Dec. 2007, 9(12):1015-23); the NXNL2/NXNL1 promoter (Lambard et al, PLoS One, Oct. 2010, 5(10):e13025), the RPE65 promoter; the retinal degeneration slow/peripherin 2 (Rds/perph2) promoter (Cai et al, Exp Eye Res. 2010 August; 91(2):186-94); and the VMD2 promoter (Kachi et al, Human Gene Therapy, 2009 (20:31-9)). Each of these documents is incorporated by reference herein in its entirety. In another embodiment, the promoter is selected from human human EF1α promoter, rhodopsin promoter, rhodopsin kinase, interphotoreceptor binding protein (IRBP), cone opsin promoters (red-green, blue), cone opsin upstream sequences containing the red-green cone locus control region, cone transducing, and transcription factor promoters (neural retina leucine zipper (Nrl) and photoreceptor-specific nuclear receptor Nr2e3, bZIP).

In another embodiment, the promoter is a ubiquitous or constitutive promoter. An example of a suitable promoter is a hybrid chicken p-actin (CBA) promoter with cytomegalovirus (CMV) enhancer elements. In another embodiment, the promoter is the CB7 promoter. Other suitable promoters include the human p-actin promoter, the human elongation factor-1α promoter, the cytomegalovirus (CMV) promoter, the simian virus 40 promoter, and the herpes simplex virus thymidine kinase promoter. See, e.g., Damdindorj et al, (August 2014) A Comparative Analysis of Constitutive Promoters Located in Adeno-Associated Viral Vectors. PLoS ONE 9(8): e106472. Still other suitable promoters include viral promoters, constitutive promoters, regulatable promoters [see, e.g., WO 2011/126808 and WO 2013/04943]. Alternatively a promoter responsive to physiologic cues may be utilized in the expression cassette, rAAV genomes, vectors, plasmids and viruses described herein. In one embodiment, the promoter is of a small size, under 1000 bp, due to the size limitations of the AAV vector. In another embodiment, the promoter is under 400 bp. Other promoters may be selected by one of skill in the art.

In a further embodiment, the promoter is selected from SV40 promoter, the dihydrofolate reductase promoter, and the phosphoglycerol kinase (PGK) promoter, rhodopsin kinase promoter, the rod opsin promoter, the red-green opsin promoter, the blue opsin promoter, the inter photoreceptor binding protein (IRBP) promoter and the cGMP-β-phosphodiesterase promoter, a phage lambda (PL) promoter, a herpes simplex viral (HSV) promoter, a tetracycline-controlled trans-activator-responsive promoter (tet) system, a long terminal repeat (LTR) promoter, such as a RSV LTR, MoMLV LTR, BIV LTR or an HIV LTR, a U3 region promoter of Moloney murine sarcoma virus, a Granzyme A promoter, a regulatory sequence(s) of the metallothionein gene, a CD34 promoter, a CD8 promoter, a thymidine kinase (TK) promoter, a B19 parvovirus promoter, a PGK promoter, a glucocorticoid promoter, a heat shock protein (HSP) promoter, such as HSP65 and HSP70 promoters, an immunoglobulin promoter, an MMTV promoter, a Rous sarcoma virus (RSV) promoter, a lac promoter, a CaMV 35S promoter, a nopaline synthetase promoter, an MND promoter, or an MNC promoter. The promoter sequences thereof are known to one of skill in the art or available publically, such as in the literature or in databases, e.g., GenBank, PubMed, or the like.

In another embodiment, the promoter is an inducible promoter. The inducible promoter may be selected from known promoters including the rapamycin/rapalog promoter, the ecdysone promoter, the estrogen-responsive promoter, and the tetracycline-responsive promoter, or heterodimeric repressor switch. See, Sochor et al, An Autogenously Regulated Expression System for Gene Therapeutic Ocular Applications. Scientific Reports, 2015 Nov. 24; 5:17105 and Daber R, Lewis M., A novel molecular switch. J Mol Biol. 2009 Aug. 28; 391(4):661-70, Epub 2009 Jun. 21 which are both incorporated herein by reference in their entirety.

Examples of suitable polyA sequences include, e.g., a synthetic polyA or from bovine growth hormone (bGH), human growth hormone (hGH), SV40, rabbit β-globin (RGB), or modified RGB (mRGB).

Examples of suitable enhancers include, e.g., the CMV enhancer, the RSV enhancer, the alpha fetoprotein enhancer, the TTR minimal promoter/enhancer, LSP (TH-binding globulin promoter/alpha1-microglobulin/bikunin enhancer), an APB enhancer, ABPS enhancer, an alpha mic/bik enhancer, TTR enhancer, en34, ApoE amongst others.

As used in the methods described herein, “administering” means delivering a therapy to a subject for treatment of ocular disease. In one embodiment, the method involves administration via subretinal injection to the RPE, photoreceptor cells or other ocular cells. In one embodiment, the method involves administration via subretinal injection to the RPE. In another embodiment, intravitreal injection to ocular cells is employed. In still another method, injection via the palpebral vein to ocular cells may be employed. In still another embodiment, suprachoroidal injection to ocular cells may be employed. Still other methods of administration may be selected by one of skill in the art given this disclosure. By “administering” or “route of administration” is delivery of a therapy described herein (e.g. a rAAV comprising a nucleic acid sequence encoding BEST1), with or without a pharmaceutical carrier or excipient, of the subject. Routes of administration may be combined, if desired. In some embodiments, the administration is repeated periodically. Direct delivery to the eye (optionally via ocular delivery, subretinal injection, intra-retinal injection, intravitreal, topical), or delivery via systemic routes, e.g., intraarterial, intraocular, intravenous, intramuscular, subcutaneous, intradermal, and other parental routes of administration. In certain embodiments, the methods provide herein include administration of nucleic acid molecules and/or vectors described herein in a single composition or multiple compositions. Optionally, two or more different AAV may be delivered, or multiple viruses [see, e.g., WO20 2011/126808 and WO 2013/049493]. In another embodiment, multiple viruses may contain different replication-defective viruses (e.g., AAV and adenovirus), alone or in combination with proteins.

As used herein, the term “ocular cells” refers to any cell in, or associated with the function of, the eye. The term may refer to any one of photoreceptor cells, including rod, cone and photosensitive ganglion cells or retinal pigment epithelium (RPE) cells. In one embodiment, the ocular cells are the photoreceptor cells. In another embodiment, the ocular cells are the RPE.

Also provided herein are pharmaceutical compositions. The pharmaceutical compositions described herein are designed for delivery to subjects in need thereof by any suitable route or a combination of different routes. These delivery means are designed to avoid direct systemic delivery of the suspension containing the AAV composition(s) described herein. Suitably, this may have the benefit of reducing dose as compared to systemic administration, reducing toxicity and/or reducing undesirable immune responses to the AAV and/or transgene product.

In yet other aspects, these nucleic acid sequences, vectors, expression cassettes and rAAV viral vectors are useful in a pharmaceutical composition, which also comprises a pharmaceutically acceptable carrier, excipient, buffer, diluent, surfactant, preservative and/or adjuvant, etc. Such pharmaceutical compositions are used to express BEST1 in the host cells through delivery by such recombinantly engineered AAVs or artificial AAVs.

To prepare these pharmaceutical compositions containing the nucleic acid sequences, vectors, expression cassettes and rAAV viral vectors, the sequences or vectors or viral vectors are preferably assessed for contamination by conventional methods and then formulated into a pharmaceutical composition suitable for administration to the eye. Such formulation involves the use of a pharmaceutically and/or physiologically acceptable vehicle or carrier, particularly one suitable for administration to the eye.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline).

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intravitreal delivery. In one example, the composition is formulated for subretinal delivery. In another example, the composition is formulated for suprachoroidal delivery.

In the case of AAV viral vectors, quantification of the genome copies (“GC”), vector genomes (“VG”), or virus particles may be used as the measure of the dose contained in the formulation or suspension. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually the transgene or the poly A signal). In another method the effective dose of a recombinant adeno-associated virus carrying a nucleic acid sequence encoding BEST1 is measured as described in S. K. McLaughlin et al, 1988 J. Virol., 62:1963, which is incorporated by reference in its entirety.

As used herein, the term “dosage” can refer to the total dosage delivered to the subject in the course of treatment, or the amount delivered in a single unit (or multiple unit or split dosage) administration. The pharmaceutical virus compositions can be formulated in dosage units to contain an amount of replication-defective virus carrying the nucleic acid sequences encoding BEST1 as described herein that is in the range of about 1.0×10⁹ vg (vector genomes)/mL to about 1.0×10¹⁵ vg/mL including all integers or fractional amounts within the range. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹vg/mL including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ vg/mL including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ vg/mL including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² vg/mL including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ vg/mL including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ vg/mL including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ vg/mL including all integers or fractional amounts within the range. In one embodiment, for human application the dose can range from 1×10¹⁰ to about 1×10² vg/mL including all integers or fractional amounts within the range. All dosages may be measured by any known method, including as measured by oqPCR or digital droplet PCR (ddPCR) as described in, e.g., M. Lock et al, Hum Gene Ther Methods. 2014 April; 25(2):115-25. doi: 10.1089/hgtb.2013.131, which is incorporated herein by reference.

In one embodiment, an aqueous suspension suitable for administration to patient having, or suspected of having, a bestrophinopathy, is provided. The suspension comprises an aqueous suspending liquid and about 1×10⁹ GC or viral particles to about 1×10¹² GC or viral particles per eye of a recombinant adeno-associated virus (rAAV) described herein useful as a therapeutic for bestrophinopathy. In one embodiment, about 1.5×10¹⁰ GC or viral particles are administered per eye.

It may also be desirable to administer multiple “booster” dosages of the pharmaceutical compositions of this invention. For example, depending upon the duration of the transgene within the ocular target cell, one may deliver booster dosages at 6 month intervals, or yearly following the first administration. The fact that AAV-neutralizing antibodies were not generated by administration of the rAAV vector should allow additional booster administrations.

Such booster dosages and the need therefor can be monitored by the attending physicians, using, for example, the retinal and visual function tests and the visual behavior tests described in the examples below. Other similar tests may be used to determine the status of the treated subject over time. Selection of the appropriate tests may be made by the attending physician. Still alternatively, the method of this invention may also involve injection of a larger volume of virus-containing solution in a single or multiple infection to allow levels of visual function close to those found in wildtype retinas.

In another embodiment, the amount of the vectors, the virus and the replication-defective virus described herein carrying the nucleic acid sequences encoding BEST1 are in the range of about 1.0×10⁷ VG per eye to about 1.0×10¹⁵ VG per eye including all integers or fractional amounts within the range. In one embodiment, the amount thereof is at least 1×10⁷, 2×10⁷, 3×10⁷, 4×10⁷, 5×10⁷, 6×10⁷, 7×10⁷, 8×10⁷, or 9×10⁷ VG per eye including all integers or fractional amounts within the range. In one embodiment, the amount thereof is at least 1×10⁸, 2×10⁸, 3×10⁸, 4×10⁸, 5×10⁸, 6×10⁸, 7×10⁸, 8×10⁸, or 9×10⁸ VG per eye including all integers or fractional amounts within the range. In one embodiment, the amount thereof is at least 1×10⁹, 2×10⁹, 3×10⁹, 4×10⁹, 5×10⁹, 6×10⁹, 7×10⁹, 8×10⁹, or 9×10⁹VG per eye including all integers or fractional amounts within the range. In one embodiment, the amount thereof is at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ VG per eye including all integers or fractional amounts within the range. In one embodiment, the amount thereof is at least 1×10¹¹, 2×10¹¹, 3×10¹¹, 4×10¹¹, 5×10¹¹, 6×10¹¹, 7×10¹¹, 8×10¹¹, or 9×10¹¹ VG per eye including all integers or fractional amounts within the range. In one embodiment, the amount thereof is at least 1×10¹², 2×10¹², 3×10¹², 4×10¹², 5×10¹², 6×10¹², 7×10¹², 8×10¹², or 9×10¹² VG per eye including all integers or fractional amounts within the range. In one embodiment, the amount thereof is at least 1×10¹³, 2×10¹³, 3×10¹³, 4×10¹³, 5×10¹³, 6×10¹³, 7×10¹³, 8×10¹³, or 9×10¹³ VG per eye including all integers or fractional amounts within the range. In one embodiment, the amount thereof is at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ VG per eye including all integers or fractional amounts within the range. In one embodiment, the amount thereof is at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ VG per eye including all integers or fractional amounts within the range. In one embodiment, the methods comprises dose ranging from 1×10⁹ to about 1×10¹³ VG per eye per dose including all integers or fractional amounts within the range. In another embodiment, the method comprises delivery of the vector in an aqueous suspension. In another embodiment, the method comprises administering the rAAV described herein in a dosage of from 1×10⁹ to 1×10¹³ VG in a volume about or at least 150 microliters, thereby restoring visual function in said subject.

These above doses may be administered in a variety of volumes of carrier, excipient or buffer formulation, ranging from about 25 to about 1000 microliters, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is about 800 μL. In another embodiment, the volume is between about 150 and 800 μL. In another embodiment, the volume is between about 700 and 1000 μL. In another embodiment, the volume is between about 250 and 500 μL.

In one embodiment, the viral constructs may be delivered in doses of from at least 1×10⁹ to about least 1×10¹¹ GCs in volumes of about 1 μL to about 3 μL for small animal subjects, such as mice. For larger veterinary subjects having eyes about the same size as human eyes, the larger human dosages and volumes stated above are useful. See, e.g., Diehl et al, J. Applied Toxicology, 21:15-23 (2001) for a discussion of good practices for administration of substances to various veterinary animals. This document is incorporated herein by reference.

It is desirable that the lowest effective concentration of virus or other delivery vehicle be utilized in order to reduce the risk of undesirable effects, such as toxicity, retinal dysplasia and detachment. Still other dosages in these ranges may be selected by the attending physician, taking into account the physical state of the subject, preferably human, being treated, the age of the subject, the bestrophinopathy and the degree to which the disorder, if progressive, has developed.

In certain embodiments, treatment efficacy is determined by identifying an at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% improvement or change relative to a measurement in a control sample. The control sample may be a normal healthy control, a mutant disease control, a pre-treatment control, an earlier timepoint control, an untreated contralateral eye, or a retinal region outside of a treatment bleb. In certain embodiments, the mutant disease control is a sample from a subject with two mutant BEST1 alleles. In yet other embodiments, the mutant disease control is from a subject having one mutant BEST1 allele and a wildtype BEST1 allele.

In certain embodiments, provided herein are methods for evaluating a treatment for a BEST1-associated maculopathy in a subject. Accordingly, the subject harbors at least one mutant BEST1 gene. In certain embodiments, the subject is heterozygous for a BEST1 mutation (e.g., one mutant BEST1 allele and one wildtype, functional BEST1 allele or a carrier of alternative mutant BEST1 alleles). In certain embodiments, following administration of the therapy, the effectiveness of the treatment is determined by performing in vivo retinal imaging to evaluate one or more of a longitudinal reflectivity profile (LRP), IS/OS to retinal pigment epithelium (RPE) distance in light-adapted and/or dark-adapted eyes, and formation of light-potentiated subretinal microdetachments (as described, for example, in Guziewicz et al., PNAS. 2018 Mar. 20; 115 (12):E2839-E2848, which is incorporated by reference herein). These parameters can be supplemented with additional methods known in the art for evaluating visual function and severity of ocular disease. The effectiveness of the therapy is evaluated following administration of a therapy at time points selected based on factors such as the severity of disease, parameter to be measured, or age or species of the subject, or nature of the therapy. Accordingly, in certain time points, the effectiveness of treatment is evaluated one or more intervals following administration of a therapy. In certain embodiments, treatment efficacy is evaluated within 24 hours, 36 hours, 48 hours, or 72 hours following administration of a therapy. In yet further embodiments, treatment efficacy is evaluated one or more times within 1 week, 2 weeks, 1 month, 2 months, 3 months, 4 months, 5 months, or 6 months of administering a therapy. In certain embodiments, the therapy is treatment with a viral vector, as described herein.

Canine bestrophinopathy arises as a focal detachment between retinal pigment epithelium (RPE) and the neural retina in the area centralis and can stay limited to the canine fovea-like region or develop extramacular satellite lesions, manifestations parallel to BVMD and ARB phenotype in patients. The typical cBest presents bilaterally, has an early onset (˜12 weeks of age), and progresses slowly following well-defined clinical stages described in BVMD: Stage I, pre-vitelliform with a discreet disruption between the RPE and neural retina within the canine fovea-like region; Stage II, vitelliform, characterized by a circular, yolk-like central lesion; Stage III, pseudohypopyon phase, Stage IV, vitelliruptive, and finally Stage V, atrophic—all highly comparable between BVMD patients and cBest dogs.

Thus, in certain embodiments, the methods provided herein include administering a therapy to a canine animal model for bestrophinopathy, wherein the canine harbors BEST1 mutation that recapitulates clinical, molecular, and/or histological features characteristic of human disease. Suitable mutations include previously identified spontaneous mutations, such as c.73C>T/p.R25*, -c.482G>A/p.G161D, and c.1388delC/P463fs. cBEST1-C73T/R25* -contains a premature stop codon, resulting in null phenotype; cBEST1-G482A/G161D which contains a missense change, affecting protein folding and trafficking; and cBEST1-C1388del/P463fs which contains a frameshift mutation, truncating the C-terminus of bestrophin-1 protein. In certain embodiments, the canine has a wildtype BEST1 allele and a mutated BEST1 allele. The mutated BEST1 allele may have one or more mutations. Additional BEST1 mutations can be identified by one of ordinary skill in the art to generate animal models to be used in the methods describe herein.

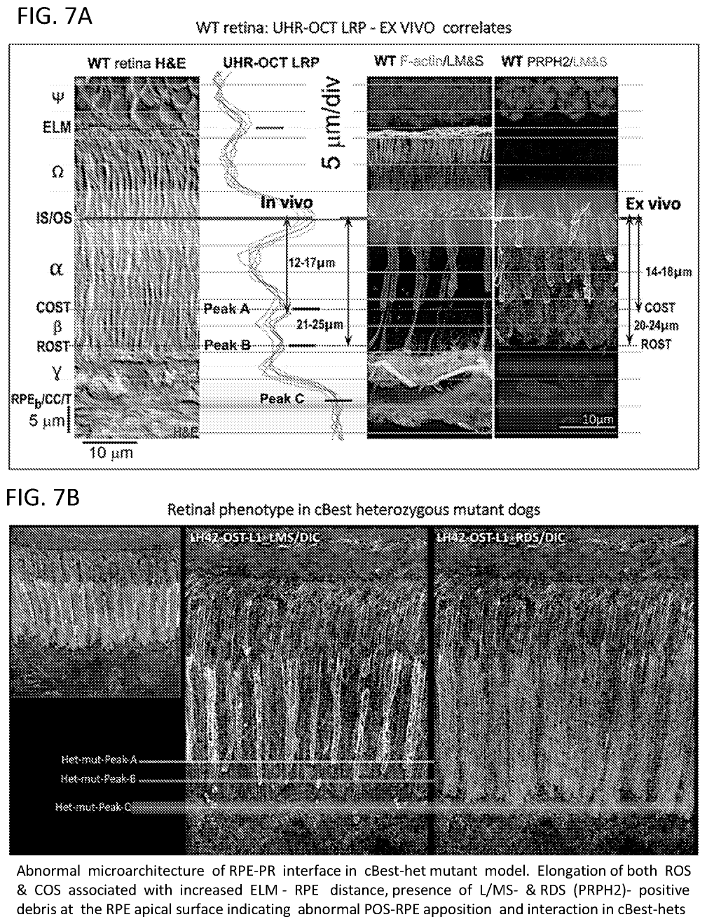

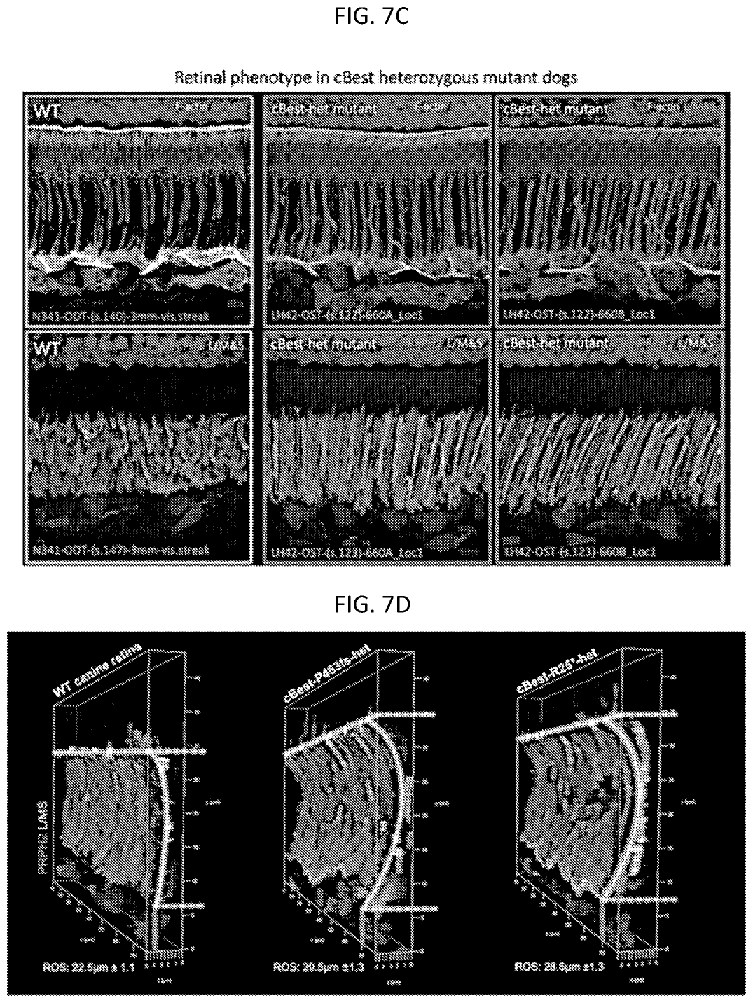

As described herein, for the first time, a previously undetected disease phenotype has been recognized in cBest heterozygote mutants. The data herein validate the cBest-heterozygous (cBEST-Het) mutant dog model. The cBest-Hets demonstrate a phenotype which shares overlapping disease aspects and pathogenesis with the cBest-homozygous mutant models previously described, but at a subtle, subclinical level. However, the subclinical manifestations observed in the cBest-Hets and described herein have not been previously identified or described, and are, identifiable only via testing with ultra-high resolution instrumentation, such as those described herein. The cBest-Het and cBest-homozygous models demonstrate retina-wide pathology of the RPE-photoreceptor interface. For example, FIGS. 7A and 7B, looking at peak C, it can be seen that the RPE-PR interface of the cBest-Het model demonstrates abnormal microarchitecture due to elongation of both ROS and COS associated with increased ELM-RPE distance, the presence of L/MS- and RDS (PRPH2)-positive debris at the RPE apical surface indicating abnormal POS-RPE apposition and interaction in cBest-Hets. Furthermore, the cBest-Hets demonstrate thinning, elongation and curving of the ROS as compared to wild type retina (FIG. 7D), as well as increased formation of debris. In addition, the cBest-Het model demonstrates dysregulation of lipid homeostasis, similar to the cBest homozygous model. It is desirable that a therapeutic treatment ameliorate one or more of these phenotypic changes. In one embodiment, the treatment reduces COS elongation, thinning, and/or curving. In another embodiment, the treatment reduces ROS elongation, thinning, and/or curving. In another embodiment, the treatment reduces glial activation. In another embodiment, the treatment reduces ELM-RPE distance, in another embodiment, treatment reduces accumulation of retinal debris. In another embodiment, treatment reduces abnormal POS-RPE apposition and microarchitecture of RPE-PR interface. In another embodiment, treatment reduces subretinal debris at RPE apical surface, or within subretinal space. In another embodiment, treatment reduces compromised IPM and defective ELM. In another embodiment, treatment reduces fluctuation of ONL thickness associated with reactive gliosis and cell migration. In another embodiment, treatment reduces schistic changes in the inner/outer retina. In another embodiment, treatment reduces formation of subretinal & intraretinal scars. In another embodiment, treatment reduces RPE monolayer hypertrophy. In another embodiment, treatment reduces occasional severe deformation of individual RPE cells associated with ONL & INL thickness fluctuations. In another embodiment, treatment reduces and Muller Glial trunks/projections penetrating ONL layer.

In certain embodiments of the invention it is desirable to perform non-invasive retinal imaging and functional studies to identify areas of the rod and cone photoreceptors to be targeted for therapy. In certain embodiments, clinical diagnostic tests are employed to determine the precise location(s) for one or more subretinal injection(s). These tests may include electroretinography (ERG), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc, depending upon the species of the subject being treated, their physical status and health and treatment.

In certain embodiments, the methods include generating a longitudinal reflectivity profile (LRP) using an optical coherence tomography (OCT) system. In certain embodiments, imaging of the retina is performed using an ultrahigh-resolution OCT (UHR-OCT) system, such as the Leica/Bioptigen Envisu OCT System or a system capable of similar high-resolution imaging). See, e.g., FIG. 7A demonstrating a LRP generated using an UHR-OCT system. In certain embodiments, ultrahigh resolution OCT is essential to generate a LRP used to evaluate a retinal phenotype. Accordingly, standard imaging systems (e.g., Spectralis HRA +OCT) are not sufficient to reveal retinal phenotypes for purposes of certain methods described herein. In certain embodiments, the LRP is further evaluated to assess parameters that indicate the effectiveness of a treatment. The effectiveness of a treatment can be evaluated, for example, based on examining cytoarchitecture at the RPE-photoreceptors (PRs) interface apposition between RPE and PRs. In certain embodiments, in vivo imaging is used to evaluate the extent of retina-wide RPE-PR macro- or microdetachment to determine the effectiveness of a treatment.

As described herein, and as discussed in the Examples below, the UHR-OCT LRP and generated LRP show the length of cone outer segments (IS/OS to cone outer segment tip (COST) as shown in FIG. 7A, Peak A) and length of rod outer segments (IS/OS to rod outer segment tip (ROST) as shown in FIG. 7A, Peak B) correlate with both in vivo and ex vivo histological analysis. See, e.g., FIG. 7A. Further, the cBest-Hets show elongation of the cone outer segments and rod outer segments. Further, as demonstrated in FIGS. 7A and 7B, cBest model demonstrates abnormal microarchitecture of the RPE-PR interface. These described changes are measurable in both the cBest models, and subject patients. These measurements can be used to help determine efficacy of treatment, as well as identification of subjects requiring medical intervention for Best disease.

In certain embodiments, the COS and/or ROS are evaluated to determine if lengthening is present. In one embodiment, a COS measurement of greater than about 12 μm to about 17 μm is indicative of Best disease. In some embodiments, a COS measurement of greater than about 10, 11, 12, 13, 14, 15, 16, 17, 18, 19, or 20 μm is indicative of Best disease.

In one embodiment, a ROS measurement of greater than about 20 μm to about 27 μm is indicative of Best disease. In some embodiments, a ROS measurement of greater than about 20, 21, 22, 23, 24, 25, 26, 27, 28, 29, or 30 μm is indicative of Best disease.

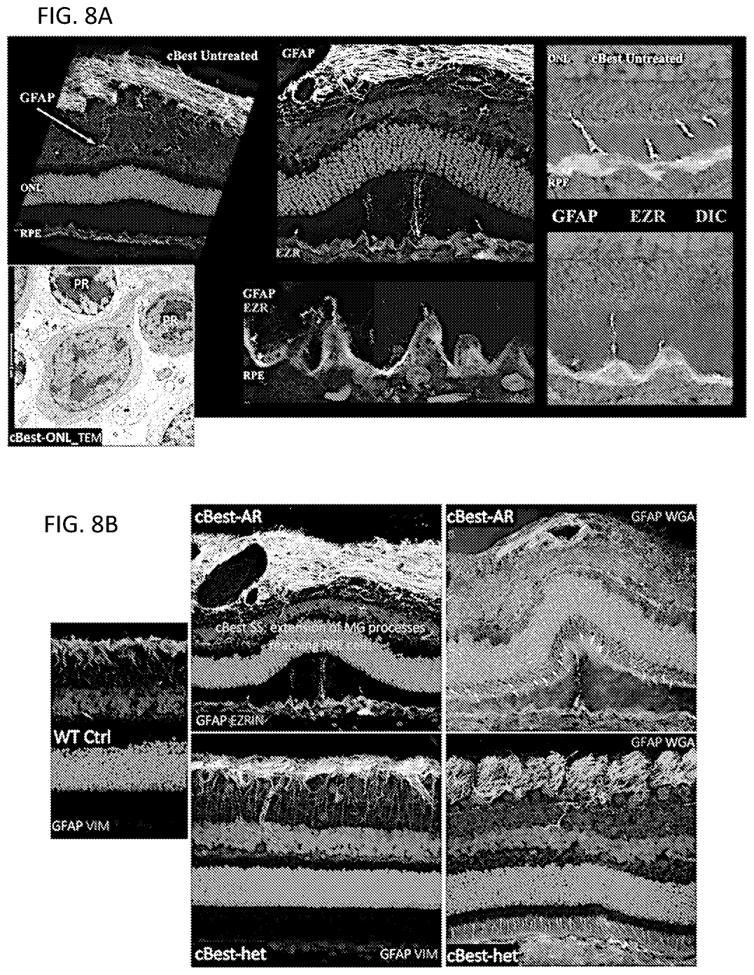

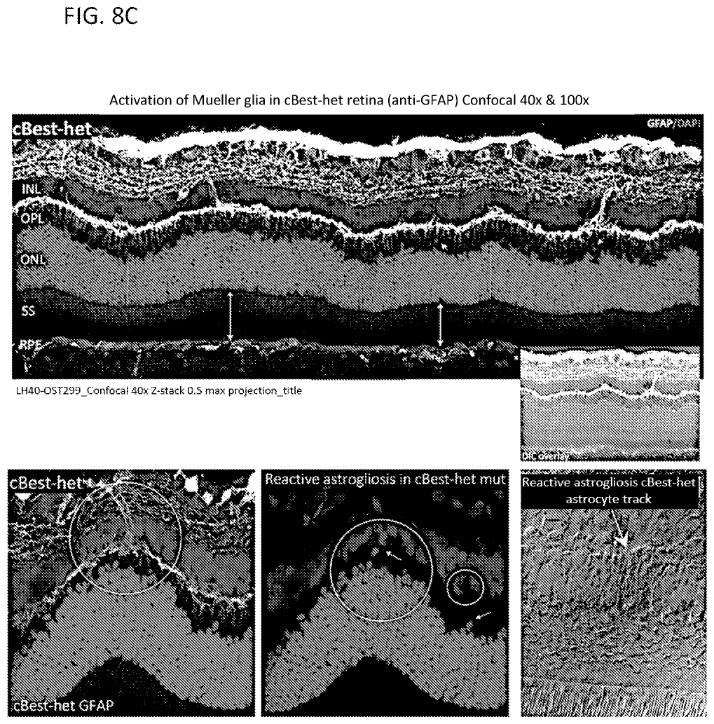

As demonstrated herein, gliotic changes are a hallmark of Best disease, in both the autosomal dominant and autosomal recessive disease. The gliotic changes are a result of constant insult and inflammation to the retina and are observed, inter alia, as Muller glia (MG) trunks or projections penetrating the ONL layer. For example, in FIGS. 8A and 8B it can be seen that the MG processes reach the RPE in the cBest-Het model.

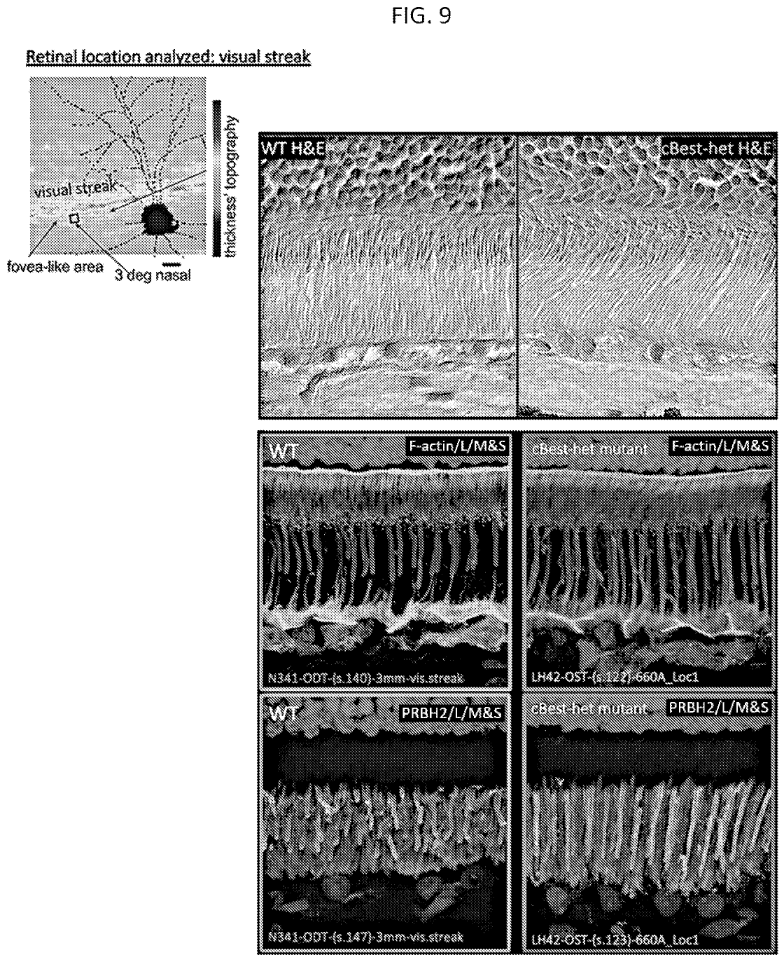

In one embodiment, retinal changes indicative of Best-1 disease include one or more of abnormal POS-RPE apposition and microarchitecture of RPE-PR interface (FIG. 7B); Elongation of both ROS & COS associated with increased ELM-RPE distance (FIG. 7B-FIG. 7D, FIG. 9); Accumulation of subretinal debris at RPE apical surface (FIG. 9), within subretinal space (FIG. 7B-FIG. 7D); Compromised IPM and defective ELM; Fluctuation of ONL thickness associated with reactive gliosis and cell migration; Schistic changes inner/outer retina; Formation of subretinal & intraretinal scars; RPE monolayer hypertrophy, occasional severe deformation of individual RPE cells associated with ONL & INL thickness fluctuations; MG trunks/projections penetrating ONL layer with astrogliosis as an indicator of chronic retinal stress (FIG. 8B).

In certain embodiments, provided herein are methods for detecting an autosomal dominant BEST1 mutation or diagnosing a subject as having autosomal dominant bestrophinopathy. In certain embodiments, the method includes performing retinal imaging using ultrahigh-resolution OCT to generate a longitudinal reflectivity profile (LRP), wherein an abnormal RPE-PR interdigitation zone results in an altered LRP profile indicating that the subject harbors an autosomal dominant BEST1 mutation.

In certain embodiments, the methods provided herein include obtaining a sample from a treated subject for examination ex vivo. Accordingly, an ocular tissue sample is examined by labeling with reagents that bind ocular cells and/or markers in the sample to evaluate a phenotype. The sample may be analyzed, for example, using fluorescence microscopy or immunohistochemistry. In certain embodiments, retinal lesions in a sample are evaluated for accumulation of autofluorescent material in RPE cells or the subretinal space. In yet other embodiments, the sample is evaluated to determine cytoskeletal rescue and restoration of restoration of RPE apical microvilli structure, a reestablishment of proper apposition between RPE cells and photoreceptor (PR) outer segments (cytoarchitecture of RPE-PR interface), and/or a restoration of the insoluble cone-specific interphotoreceptor matrix (IPM) to determine treatment efficacy (as described, for example, in Guziewicz et al., PLoS One. 2013 Oct. 15; 8 (10):e75666 and Guziewicz et al, PNAS. 2018 Mar. 20; 115 (12):E2839-E2848, each of which is incorporated by reference herein). In certain embodiments the sample is labeled with reagents that bind one or more of BEST1, RPE65, EZRIN, pEZRIN, MCT1, CRALBP, F-actin, hCAR, an L-opsin, an M-opsin, an S-opsin, and RHO.

The following examples are provided for the purpose of illustration only and the invention should in no way be construed as being limited to these examples but rather should be construed to encompass any and all variations that become evident as a result of the teaching provided herein.

Described herein is a sub-clinical phenotype in a canine cBest disease model associated with abnormal microarchitecture of RPE-PR interface and expose retinal pathways leading to chronic retinal stress, reactive Muller cells' gliosis and astrocytosis, both contributing to neuronal dysfunction in mono allelic BEST1 disease. Our findings support that these sub-clinical abnormalities are amenable to AAV-mediated BEST1 gene augmentation therapy, expanding the therapeutic landscape for Best patients.

The cBest-Het mutant model demonstrates various disease features which are observable by the skilled artisan including: Abnormal POS-RPE apposition and microarchitecture of RPE-PR interface; Elongation of both ROS & COS associated with increased ELM-RPE distance; Accumulation of subretinal debris at RPE apical surface, within subretinal space; Compromised IPM and defective ELM similar to UHR findings in human Best disease; Fluctuation of ONL thickness associated with reactive gliosis and cell migration; Schistic changes inner/outer retina; Formation of subretinal & intraretinal scars; RPE monolayer hypertrophy, occasional severe deformation of individual RPE cells associated with ONL & INL thickness fluctuations; MG trunks/projections penetrating ONL layer with astrogliosis as an indicator of chronic retinal stress.

### EXAMPLES

### Example 1: Methods

cBest Dogs

All cBest-mutant and control dogs are bred and maintained at the Retinal Disease Studies Facility (RDSF), Kennett Square, Pa., USA. The studies are carried out in strict accordance with the recommendations in the Guide for the Care and Use of Laboratory Animals of the of the National Institutes of Health (NIH), and in compliance with the Association for Research in Vision & Ophthalmology (ARVO) Statement for the Use of Animals in Ophthalmic and Vision Research. The protocols were approved by the Institutional Animal Care and Use Committee of the University of Pennsylvania (IACUC #s 804956, 803422). All efforts are made to improve animal welfare and minimize discomfort.

### Genotyping

The genotypes of cBest dogs are determined using previously developed PCR-based assays with canine BEST1 (cBEST1) (GB #NM_001097545.1) gene specific primers (Guziewicz et al., 2007; Zangerl et al., 2010). To confirm cBEST1 heterozygous mutations (c.73C>T or c.482G>A or c.1388delC), PCR amplicons are purified (ExoSAP-IT, ThermoFisher Scientific, Waltham, Mass., USA), submitted for direct Sanger sequencing (UPenn NAPCore Facility, The Children's Hospital of Philadelphia, Pa., USA), and analyzed with the use of Sequencher v.5.2.4 software package (Gene Codes, Ann Arbor, Mich., USA).

### Ophthalmic Examination and In Vivo Retinal Imaging

Ophthalmic examinations, including biomicroscopy, indirect ophthalmoscopy and fundus photography, are conducted on a regular basis, starting at 5 weeks of age, then biweekly before cSLO/OCT baseline evaluation, and every 4 weeks thereafter.

Non-invasive retinal imaging in cBest-mutant and control dogs is performed under general anesthesia after pupillary dilation and conducted according to methods similar to previously described (Cideciyan et al., 2005; Beltran et al., 2012; Guziewicz et al., 2018). Overlapping en face images of reflectivity with near-infrared illumination (820 nm) are obtained with 300 and 550 diameter lenses (Spectralis HRA+OCT, Heidelberg, Germany) to delineate fundus features such as optic nerve, retinal blood vessels, retinotomy post subretinal injection or other local changes. Custom programs (MatLab 7.5; The MathWorks, Natick, Mass., USA) are used to digitally stitch individual photos into a retina-wide panorama. Imaging with an ultrahigh-resolution OCT system (Leica/Bioptigen)

Retinal cross-sectional images of cBest and control eyes were acquired with an Envisu R2210 UHR (Ultra-High Resolution) SD-OCT system (Bioptigen, Leica Microsystems, Morrisville, N.C., USA) with methods similar to previously described (Aleman et al., 2011; Huang et al., 2012; Boye et al., 2014). ‘Rabbit’ lens was used, and the angular magnification was adjusted by matching features visible on the same canine eye scanned with Spectralis as well as Bioptigen/Envisu systems. The retinal location of interest centered at the canine fovea-like region was found under fast fundus mode. High-resolution scans (100 parallel raster scans of 1000 LRP each repeated three times) were acquired at this location. Each LRP had 1024 samples representing 1654 μm of retinal depth along the z-axis (1.615 μm/sample). Post-acquisition processing of OCT data was performed with custom programs (MatLab 7.5; The MathWorks, Natick, Mass., USA). The LRPs of the OCT images were aligned by manually straightening the Bruch's membrane (BrM) and choriocapillaris (ChC) reflection. Thickness of the outer nuclear layer (ONL) was measured between the signal peaks defining the OPL and outer limiting membrane (OLM). Number of hyper-scattering peaks were identified between the IS/OS peak and the RPE/Tapetum (RPE/T) peak, and distance between the peaks was quantified.

### Ex Vivo Assessments

The retinal microarchitecture of cBest-Het eyes is studied in comparison to the wild-type controls with assessments methods similar to previously described (Beltran et al., 2006; Guziewicz et al., 2017; Guziewicz et al., 2018).

### Histological and Immunohistochemical Evaluations

R Ocular tissues for ex vivo analyses are collected as described previously (Beltran et al., 2006; Beltran et al., 2014). The eyes are fixed in 4% paraformaldehyde or frozen, embedded in Optimal Cutting Temperature (OCT) media and processed as previously reported (Beltran et al., 2006; Guziewicz et al., 2017). Histological assessments using hematoxylin/eosin (H&E) staining, and immunohistochemical (IHC) experiments are performed on 10 μm-thick cryosections following established protocols (Beltran et al., 2006; Guziewicz et al., 2013; Guziewicz et al., 2017). Briefly, retinal cryosections are permeabilized with 1×PBS/0.25% TX-100, blocked for 1 hour at room temperature, and incubated overnight with a primary antibody. A set of RPE- and photoreceptor-specific markers (including BEST1, RPE65, EZRIN, pEZRIN, MCT1, CRALBP, F-actin, hCAR, L/M&S opsins, and RHO) is used to assay the RPE-photoreceptor interdigitation zone in cBest-Het and control retinas. For simultaneous assessment of the insoluble interphotoreceptor matrix (IPM), multicolor labeling is applied and primary antibodies combined with WGA-AF594 or PNA-AF647 (L32460; Molecular Probes, Eugene, Oreg., USA), followed by 1 hour incubation with a corresponding secondary antibody (Alexa Fluor®, ThermoFisher Scientific). The slides are examined by epifluorescence or transmitted light microscopy (Axioplan; Carl Zeiss Meditec GmbH Oberkochen, Germany), and digital images collected with a Spot4.0 camera (Diagnostic Instruments, Sterling Heights, MI, USA).

### Confocal Microscopy & Image Analysis

Microscopic images are acquired on a Leica TCS-SP5 Confocal Microscope System or Leica DM6000B Upright Microscope with DIC (Differential Interference Contrast) optics and DMC-2900 color camera (Leica Microsystems, Mannheim, Germany). To obtain high-resolution confocal photomicrographs, image stacks are acquired at 0.25 μm Z-steps with digital resolution of 2048×2048, then deconvolved with Huygens Deconvolution Software v.17.04 (Scientific Volume Imaging Inc., Hilversum, Netherlands). All deconvolved images are rendered in the Leica LAS X 3D-rendering module, and cone-associated RPE apical microvilli assessed from the maximum projection images. Data are analyzed and quantified using Prism software v.7 (Prism; GraphPad, San Diego, Calif., USA).

### Example 2: Assessment of Retinal Phenotype in cBest Heterozygous Dogs

The goal of this study was to determine whether cBest heterozygous mutant dogs (cBest-Het) present a milder disease phenotype, which would support the use of the cBest-Het model for preclinical assessment of AAV2-BEST1 gene augmentation therapy for the autosomal dominant form of the disease. Accordingly, retinal imaging with an ultrahigh-resolution OCT system (Leica/Bioptigen) was performed to determine the presence of structural abnormalities at the RPE/PR interface below the resolution of the standard clinical systems (Spectralis HRA+OCT). cBest dogs (n=9; both sexes) harboring cBEST1−cmr1: c.73C>T/p.R25* or cmr2: -c.482G>A/p.G161D or cmr3: -c.1388delC/p.P463fs mutations in heterozygous state were evaluated.

The cBest heterozygous mutant dogs were bred at the UPenn RDSF, and housed under bright light (450 lux) cyclic conditions. Retinal phenotype was monitored at baseline (12-wks of age) and followed on a 6-wk basis by ophthalmoscopy and cSLO/SD-OCT using established protocols of incremental light exposure. Imaging with an ultrahigh-resolution OCT system (Bioptigen) was performed to determine the existence of structural abnormalities below the resolution of the standard clinical systems (Spectralis). Retinal pathology was assessed at 24-wks of age (Grp1 n=3) or at 36-wks of age (Grp2 n=3). Based on the intermediate phenotype in cBest-Het dogs identified at 24- or 36-wks of age, the remaining cBest-Het group (Grp3 n=3) was kept to test correction by gene therapy (Example 3). EX vivo findings in the cBest-Het model indicate partial underdevelopment of the cytoskeleton associated with RPE apical aspect and RPE/PR interface, and suggest haploinsufficiency as the underlying cause of cBest-Het subclinical manifestation.

Serial in vivo imaging using targeted light exposure was used to determine the association between the milder cBest-Het phenotype and its sensitivity to light (light-potentiated formation of subretinal microdetachment quantified based on IS/OS-RPE/T distance measurements). Histology/IHC inform identified retinal morphological and molecular defects at the RPE-PR interdigitation zone in cBest-Het retinas. Expression of Ca-dependent molecules involved in Best1 pathway, accumulation of lipofuscin, and cytoarchitecture of RPE apical aspect (cone-MV quantification) were also examined. The characterization of cBest-Het mutant phenotype has yielded insight into the BEST1 haploinsufficiency mechanisms, and consequently, set the stage for gene augmentation therapy in patients affected with autosomal dominant bestrophinopathy.

Briefly, cmr1 mutation results in a premature stop codon in the first coding exon of cBEST1 gene, and no gene product (bestrophin-1 protein) was detected; cmr2 change is a point mutation (aka missense) in exon 5 resulting in amino acid substitution (Glycine residue ‘G’ to a polar, negatively charged Aspartic Acid ‘D’), leading to protein misfolding/ER retention/mistrafficking; cmr3 microdeletion (C1388del) initiates Pro463fs frameshift that results in a stop codon at amino acid 490 and protein truncation. All three cBEST1 mutations are naturally-occurring and lead to a highly consistent in vivo phenotype.

The cBest-Het mutant model demonstrates various disease features which are observable by the skilled artisan including: Abnormal POS-RPE apposition and microarchitecture of RPE-PR interface (FIG. 7B); Elongation of both ROS & COS associated with increased ELM-RPE distance (FIG. 7B-FIG. 7D, FIG. 9); Accumulation of subretinal debris at RPE apical surface (FIG. 9), within subretinal space (FIG. 7B-FIG. 7D); Compromised IPM and defective ELM supporting UHR findings in human Best disease; Fluctuation of ONL thickness associated with reactive gliosis and cell migration; Schistic changes inner/outer retina; Formation of subretinal & intraretinal scars; RPE monolayer hypertrophy, occasional severe deformation of individual RPE cells associated with ONL & INL thickness fluctuations; MG trunks/projections penetrating ONL layer with astrogliosis as an indicator of chronic retinal stress (FIG. 8B).

### Example 3: AAV-Mediated BEST1 Gene Augmentation

cBest-Het dogs (n=6) with established disease phenotype (Grp3, as described in Example 2) are injected unilaterally (n=6 eyes; age: 36-wks) with research-grade AAV-hBEST1 therapeutic vector (3.0E+11 vg/mL) targeting retinal areas previously exposed to the incremental light intensities. The contralateral eyes and retinal regions outside of the treatment bleb serve as controls. Treatment responses are monitored in vivo (fundus eye examination, cSLO, Bioptigen OCT) at 6-, 12-, and 24-wks post injection (p.i.), and assessed ex vivo 24-wks p.i. The reversal of the intermediate cBest-Het mutant phenotype provides baseline for determination of efficacy of correction relevant to a major proportion of patients affected with autosomal dominant form of bestrophinopathy.

### Example 4: Preclinical Assessment of AAV-BEST1 Vector

The purpose of this study is to assess outcome measures, such as retinal preservation, vector tropism, and transgene expression resulting from administration of AAV-BEST1 vector in wildtype dogs for overexpression of BEST1 protein.

Pre-dosage: physical and eye examinations (n=12 dogs); 4 dose groups; 3 dogs/dose group. Subretinal injection (MedOne kit 25G/38G cannula) (150 uL) in one eye of 12 wild-type (WT) dogs with one of 3 vector doses (High-Dose: 3×10¹² vg/mL, Mid-Dose: 3×10¹¹ vg/mL, or Low-Dose: 3×10¹⁰ vg/mL), or vehicle. Termination at 10-wks post-dosage.

In vivo outcome measures of safety:
Physical examination at pre-dosage, wk1, then at termination (wk10).
Ocular examinations at pre-dosage, day1 and day2 post injection (p.i.), then weekly until termination at wk10.
Serum collection for AAV2 neutralizing antibody titration at pre-dosage, wk1 and wk6 p.i., then at termination (wk10).
cSLO/SD-OCT examination 10-wks post injection (end-evaluation wk10) and qualitative analysis.

Ex vivo outcome measures: assessment of retinal preservation, vector tropism, and transgene expression: Retinal histology (H&E)/IHC (BEST1 transgene expression, phosphorylated Ezrin (pEzrin) qualitative analysis) in treated vs non-treated areas of injected eyes at 10-wks p.i.

### Example 5: GLP-Like Dose Range Finding/Non-Clinical Toxicology Study

Purpose: To determine under GLP-like conditions the range of efficacious doses of research-grade AAV2-hVMD2-hBEST1 vector and evaluate its safety profile.

Subjects: cBest homozygous dogs.

Study Duration: In life: 12 wks (injection at ˜12-wks of age, termination at ˜24-wks of age).

Methods: 4 dose groups. Subretinal injection (150 uL) in one eye of cBest homozygous mutant dogs at ˜12-wks of age with one of 3 vector doses (High-Dose: 3×10¹² vg/mL, Mid-Dose: 3×10¹¹ vg/mL, or Low-Dose: 3×10¹⁰ vg/mL), or vehicle. Termination at 12 weeks post-dosage.

Outcome measures of efficacy:
Assessment of retinal structure by cSLO-OCT at pre-dosage and before termination (˜12 weeks post dosage).
Retinal histology (H&E) and IHC for BEST1 transgene expression and cone MV structure in treated vs nontreated areas of ipsilateral and contralateral eyes.

Outcome measures of safety:
Physical examination (incl. body weights) at pre-dosage, wk1, then weekly until termination (wk12).
Ocular examinations at pre-dosage, wk1, then monthly until termination at wk12.
Clinical pathology (CBC, Chemistry panel, Coagulation profile) at pre-dosage, then monthly until termination at wk12.
Whole blood collection (for biodistribution studies to be coordinated by Sponsor) at pre-dosage, wk1, then monthly until termination at wk12.
Serum collection (for AAV2 Nab testing to be coordinated by Sponsor) at pre-dosage, wk1, then monthly until termination at wk12.
Full necropsy, histopathology analysis, tissue collection for biodistribution studies.

Eye examinations: at pre-dose phase, and day 3-, weeks: 1-, 2-, 4-, 8-, and 12-post-injection. cSLO/SD-OCT examination: at pre-dose and 12 wks p.i. Retinal histology (H&E)/IHC (BEST1 transgene expression; cone-MV structure) in treated vs non-treated areas. Outcomes: This study will determine range of effective and safe doses that will guide the design of a first-in-human clinical trial.

### Example 6: BVMD: Natural History & Development of Outcome Measures for AAV-BEST1 Clinical Trial

Purpose: To determine retina-wide distribution of structural and functional defects in patients with autosomal dominant Best Vitelliform Macular Degeneration (BVMD). Comparison of human dominant disease phenotype to canine recessive and dominant disease phenotype stages. Development of outcome measures for human clinical trials of focal gene therapy for BVMD.

Subjects: Patients with BVMD (n=15).

Study Duration: 18 months.

Methods: A combination of retrospective and prospective data will be analyzed. Specific methods will include cross-sectional imaging with standard and ultra-high resolution OCTs, en face imaging with near-infrared reflectance and autofluorescence, as well as short-wavelength autofluorescence. Functional methods will include light- and dark-adapted two-color computerized perimetry as well as dark-adaptometry.

Outcomes: Distribution of rod- and cone-mediated sensitivity loss across the retina. Visual cycle kinetics at selected retinal locations. Outer and inner retinal, and RPE-associated structural abnormalities, and their relation to light exposure history. In a subset of patients, long-term natural history of disease.

### Example 7: Light-Induced Acceleration of cBest Phenotype and AAV-BEST1 Therapy in Advanced cBest Disease after Light Stimulation

Purpose: To harness the light-modulated acceleration of cBest phenotype to assess AAV-hBEST1-gene therapy in advanced disease.

Subjects: 6 cBest homozygous affected dogs (n=2/mutation).

Study Duration: In life: 48 wks (n=6).

Methods: cBest homozygous dogs will be housed under standard (120 lux; n=3) or bright light (450 lux; n=3) cyclic conditions. Both cBest homozygous-affected groups will be followed by cSLO/SD-OCT imaging at 4-wk intervals (baseline at 12-wks of age) applying established protocols of targeted light stimulation.

Ophthalmological examination will be performed on 3-wk basis and retinal phenotype documented by fundoscopy.

Disease progression rate and severity will be addressed in comparison to already collected natural cBest history data of dogs not challenged with targeted light exposure protocols, and correlated with the light preconditioning paradigm set for the two groups.

If a more advanced stage of disease is achieved in these dogs following light exposure, then cBest homozygous dogs will be injected bilaterally at 24-wks of age with research-grade AAV-hBEST1 lead therapeutic vector (3.0E+11 vg/mL). Subretinal injections will be targeted to retinal areas with advanced disease, whereas retinal regions outside of the treatment bleb will serve as internal controls. Treatment response will be monitored in vivo for the next 24 wks p.i. (6-, 12-, and 24-wks p.i.), and the phenotype rescue in all 3 distinct cBest homozygous models assessed by histology & IHC by the end-evaluation (24 wks p.i.).

Outcomes: Assessment of light-induced acceleration of cBest phenotype and its reversal will provide critical insight into the disease metrics and development of outcome measures for clinical trial.

### Example 8: ARB: Natural History & Development of Outcome Measures for AAV-BEST1 Clinical Trial

Purpose: To determine retina-wide distribution of structural and functional defects in patients with autosomal recessive bestrophinopathy (ARB). Comparison of human phenotype stages to canine phenotype stages.

Development of outcome measures for human clinical trials of focal gene therapy for ARB.

Subjects: Patients with ARB (n=5).

Study Duration: 12 months.

Methods: A combination of retrospective and prospective data will be analyzed. Specific methods will include cross-sectional imaging with standard and ultra-high resolution OCTs, en face imaging with near-infrared reflectance and autofluorescence, as well as short-wavelength autofluorescence. Functional methods will include light- and dark-adapted two-color computerized perimetry as well as dark-adaptometry.

Outcomes: Distribution of rod- and cone-mediated sensitivity loss across the retina. Visual cycle kinetics at selected retinal locations. Outer and inner retinal, and RPE-associated structural abnormalities, and their relation to light exposure history.

### Example 9: Analysis of Long-Term Stability of AAV-BEST1 Treatment in cBest

Purpose: Assessment of long-term efficacy of human BEST1 transgene expression in cBest eyes followed longitudinally.

Subjects: cBest dogs (n=10; both sexes), harboring R25*/R25* or P463fs/P463fs or R25*/P463fs cBEST1 mutations, injected with AAV2-hBEST1 (titers range: 0.5-5.0E+11 vg/mL), and followed by cSLO/SD-OCT imaging for 39-147 wks post injection (p.i.).

Methods: Comprehensive analysis of existing longitudinal in vivo imaging data and retinal histological analysis.

Assessments of cBest eyes (n=20) will involve: generation of topographic maps of ONL thickness, quantification of IS/OS-RPE/T distance, comparative analysis of clinical stages in relation to patients, evaluation of phenotype rescue (reversal of macro- and micro-detachments) based on en face and cross-sectional recordings; retinal preservation will be assayed in cryosections (H&E, IHC with RPE- and neuroretina-specific markers), and examined by confocal microscopy. Restoration of RPE-PR interface structure will be assessed qualitatively and quantitatively (number of cone-MV/mm2) vs AAV-untreated control retinas.

Outcomes: Analyses of in vivo data will assist in defining disease stages in patients sensible to approach with BEST1 gene augmentation therapy. Histology/IHC will determine dose-response relationship with regard to correction of structural alterations at the RPE-PR interface.

SCOPE: cSLO/SD-OCT: topographic maps IS/OS-RPE/T distance & ONL thickness; H&E/IHC/cBest-AR eyes n=11 AAV-hBEST1-injected vs CTRLs

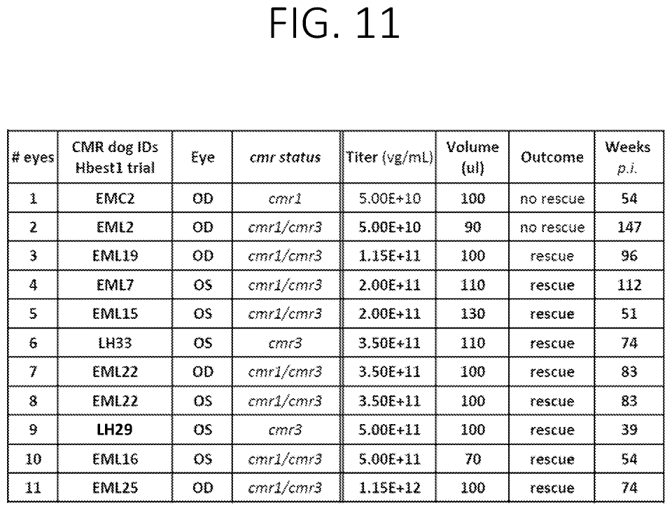

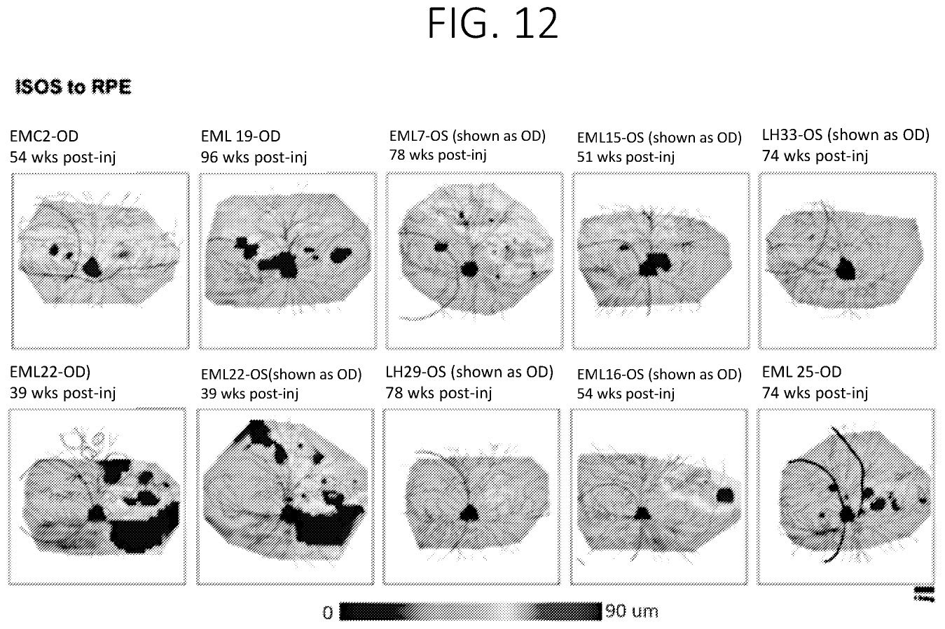

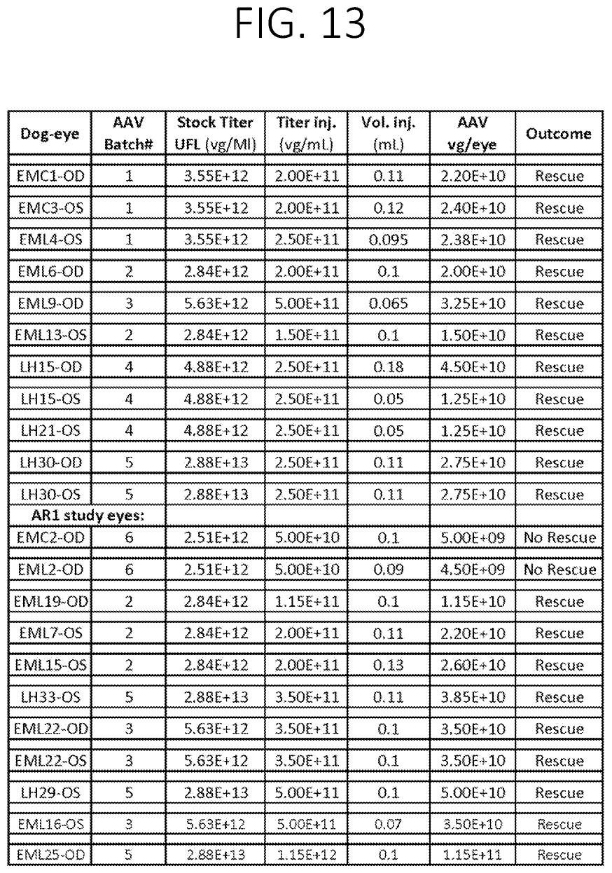

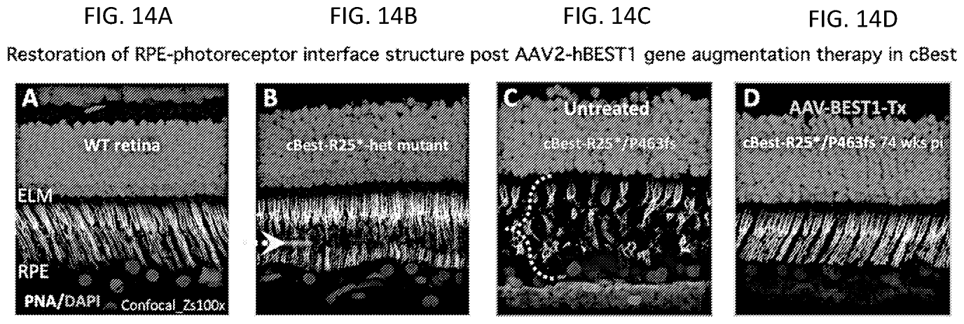

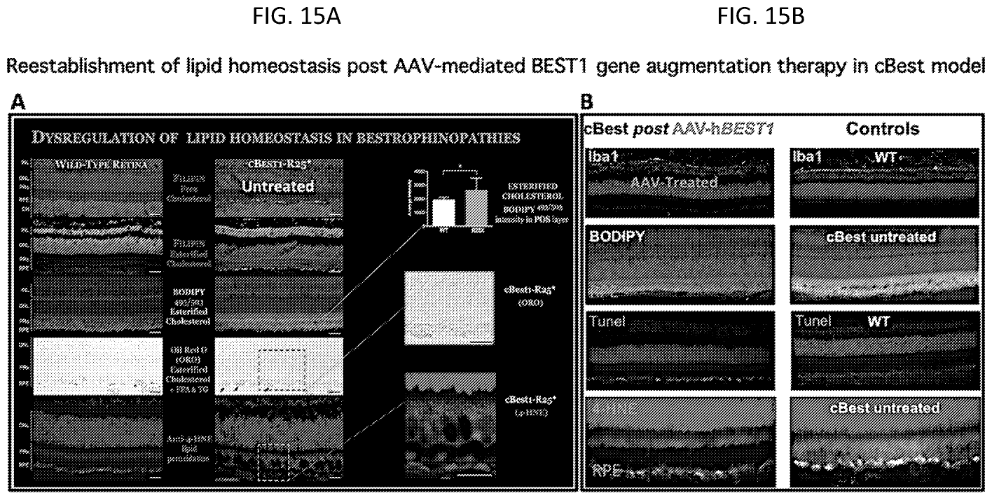

FIG. 11 shows a summary of cBest-AR rAAV2-hBest1-injected eyes enrolled in the study. All eyes receiving a dosage of 1.15×10¹¹ or higher showed rescue. FIG. 12 shows assessment of cBest-AR treated subjects up to 74 weeks post injection. FIG. 13 shows cBest eyes dosing in comparison to published cBest subjects.

### Example 10: Assessment of Treated cBest Mutant Dogs

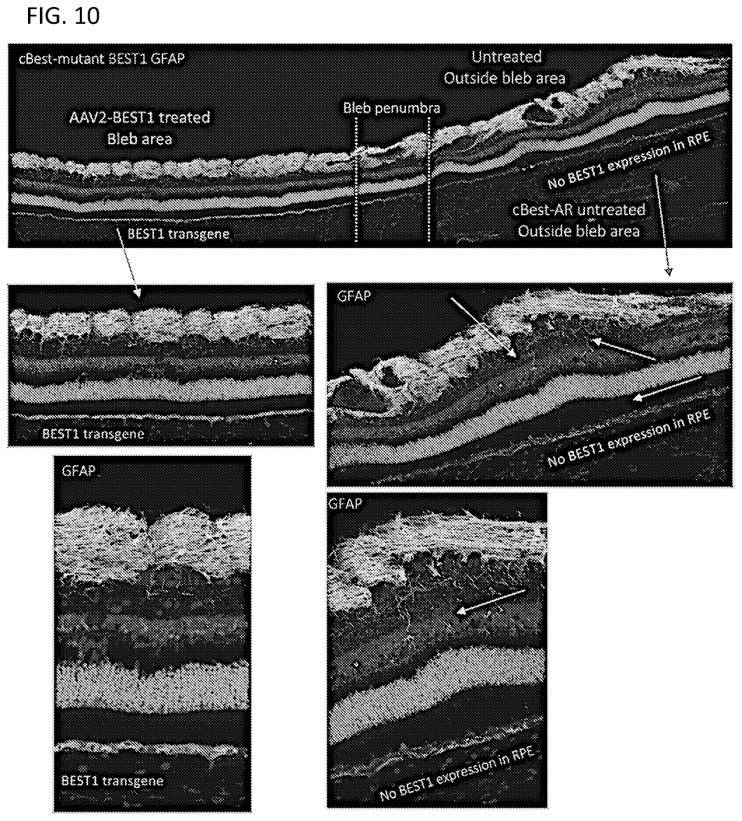

cBest mutant dogs were treated as previously described. Guziewicz et al, BEST1 gene therapy corrects a diffuse retina-wide microdetachment modulated by light exposure, Proc Natl Acad Sci USA. 2018 Mar. 20; 115 (12): E2839-E2848. Published online 2018 Mar. 5, which is incorporated herein by reference. In view of newly observed phenotypic changes in cBest-Hets described herein, treated eyes were evaluated to determine whether the gliotic changes were observable in the cBest model. Retinas were evaluated for transgene expression, and using GFAP for gliosis and astrocytosis. As previously noted, Best1 expression was observed in RPE in treated bleb area, but not outside bleb. Increased MG gliosis and astrocytosis were observed in the untreated regions (outside bleb penumbra) of treated eyes (FIG. 10), but not in AAV2-Best1 treated areas.

### Further Illustrative Embodiments

1. A method of treating a bestrophinopathy in a subject, comprising

administering to an eye of the subject a dose of a recombinant adeno-associated virus (rAAV) vector comprising a nucleic acid sequence encoding a human BEST1 protein,

wherein the subject has at least one mutant BEST1 allele, and

wherein the dose of the rAAV vector is:
a) administered at a concentration of about 1.0×10¹⁰ vector genomes (vg)/ml to about 1.0×10¹³ vg/ml; or
b) about 5.0×10⁸ vg per eye to about 5.0×10¹² vg per eye.

2. The method of embodiment 1, wherein the subject is a canine, mouse, rat, non-human primate, or human.

3. The method of embodiment 2, wherein the subject is a human.

4. The method of any one of embodiments 1 to 3, wherein the bestrophinopathy is Best Vitelliform Macular Dystrophy (BVMD), Autosomal dominant vitreoretinochoroidopathy (ADVIRC), Adult-onset vitelliform macular dystrophy (AVMD), retinitis pigmentosa (RP), or Microcornea, rod-cone dystrophy, and cataract.

5. The method of any of embodiments 1 to 4, wherein the rAAV vector is administered to the retina of the subject.

6. The method of any one of embodiments 1 to 4, wherein the rAAV vector is administered via subretinal, intravitreal, or suprachoroidal injection.

7. The method of embodiment 6, wherein the rAAV vector is administered via subretinal injection.

8. The method of any of embodiments 1 to 7, wherein the nucleic acid sequence expresses the human BEST1 protein in the retinal pigment epithelium (RPE) of the eye.

9. The method of any of embodiments 1 to 8, wherein the expression of the nucleic acid sequence encoding the BEST1 protein is under the control of a human VMD2 promoter (hVMD2).

10. The method of any of embodiments 1 to 9, wherein the dose of the rAAV vector is administered at a concentration of about 1.0×10¹⁰ vg/ml to about 3.0×10¹² vg/ml.

11. The method of embodiment 10, wherein the dose of rAAV vector is administered at a concentration of about 1.5×10¹⁰ vg/ml.

12. The method of any of embodiments 1 to 9, wherein the dose of rAAV vector is administered at a concentration of about 1.0×10¹¹ vg/ml to about 7.5×10¹¹ vg/ml.

13. The method of embodiment 12, wherein the dose of rAAV vector is administered at a concentration of about 3.0×10¹¹ vg/ml.

14. The method of embodiment 12, wherein the dose of rAAV vector is administered at a concentration of about 6.0×10¹¹ vg/ml.

15. The method of any of embodiments 1 to 9, wherein the dose of rAAV vector is administered at a concentration of about 7.5×10¹¹ vg/ml to about 1.0×10¹³ vg/ml.

16. The method of embodiment 15, wherein the dose of rAAV vector is administered at a concentration of about 3.5×10¹² vg/ml.

17. The method of any one of embodiments 1 to 16, wherein the dose of rAAV vector is administered in a volume of between about 50 ul and 500 ul.

18. The method of embodiment 17, wherein the dose of rAAV vector is administered in a volume of about 150 ul.

19. The method of embodiment 17, wherein the dose of rAAV vector is administered in a volume of about 300 ul.

20. The method of embodiment 17, wherein the dose of rAAV vector is administered in a volume of about 500 ul.

21. The method of any of embodiments 1 to 20, wherein the dose of rAAV vector administered is about 5.0×10⁸ vg per eye to about 1.5×10¹⁰ vg per eye.

22. The method of embodiment 21, wherein the dose of rAAV vector administered is about 7.5×10⁸ vg per eye.

23. The method of any of embodiments 1 to 20, wherein the dose of rAAV vector administered is about 1.0×10¹⁰ vg per eye to about 1.0×10¹¹ vg per eye.

24. The method of embodiment 23, wherein the dose of rAAV vector administered is about 4.5×10¹⁰ vg per eye.

25. The method of any of embodiments 1 to 20, wherein the dose of rAAV vector administered is about 1.0×10¹¹ vg per eye to about 5.0×10¹² vg per eye.

26. The method of embodiment 25, wherein the dose of rAAV vector administered is about 1.0×10¹² vg per eye.

27. The method of any one of embodiments 1 to 26, wherein the rAAV vector comprises an AAV2, AAV3, AAV4, AAV5, AAV6, AAV7, AAV8, AAV9, AAV10, AAV11, AAV12, LK01, LK02, LK03, AAV 4-1, AAV-2i8, Rh10, and/or Rh74 capsid, or a hybrid, chimera, or combination thereof.

28. The method of embodiment 27, wherein the rAAV vector comprises an AAV2 capsid, or a hybrid, chimera, or combination thereof.

29. The method of embodiment 28, wherein the rAAV vector comprises an AAV2 capsid.

30. The method of embodiments 29, wherein the rAAV vector is an AAV2-hVMD2-hBEST1 vector.

31. The method of any of embodiments 1 to 30, wherein the dose of rAAV is administered to each eye of the subject.

32. The method of any of embodiments 1 to 30, wherein the dose of rAAV is administered to one eye of the subject.

33. The method of embodiment 1 to 32, wherein the method does not further comprise administration of a nucleic acid composition that suppresses the expression or activity of the at least one mutant BEST1 allele.

34. The method of any of embodiments 1 to 33, wherein treatment of the bestrophinopathy is evaluated comprising:

performing in vivo retinal imaging to evaluate one or more of a longitudinal reflectivity profile (LRP), IS/OS to retinal pigment epithelium (RPE) distance in light-adapted and/or dark-adapted eyes, electrophysiology, dark-adapted kinetic perimetry and formation of light-potentiated subretinal microdetachments,

wherein treatment efficacy is indicated by one or more of a rescue of retinal microarchitecture through restoration of RPE apical microvilli structure, and a reestablishment of proper apposition between RPE cells and photoreceptor (PR) outer segments (cytoarchitecture of RPE-PR interface).

35. The method of embodiment 34, wherein the performing in vivo retinal imaging comprises one or more of fundus examination, cSLO/SD-OCT, measurement of rod outer segments, cone outer segments, ONL thickness, and ELM-RPE distance.

36. The method of embodiment 34, wherein the performing in vivo retinal imaging comprises evaluation for reactive gliosis and/or cell migration.

37. The method of embodiment 34, further comprising evaluation for Muller glial trunks/projections penetrating ONL layer with astrogliosis.

38. The method of any one of embodiments 34 to 37, wherein said retinal imaging is performed using an ultrahigh-resolution optical coherence tomography (OCT) to generate said LRP.

39. The method of any one of embodiments 34 to 38, further comprising comparing a measurement of a selected parameter to a measurement in a normal control, mutant disease control, pre-treatment control, earlier timepoint control, an untreated contralateral eye, or a retinal region outside of a treatment bleb.

40. The method of any one of embodiment 34 to 39, further comprising obtaining a retina sample from the treated subject and
a) labeling the sample with at least one RPE- and/or photoreceptor-specific marker;
b) obtaining high-resolution confocal or wide-field fluorescence microscope with Differential Interference Contrast (DIC) option images of the RPE-PR interdigitation zone; and
c) assessing one or more of length of RPE apical microvilli, structure of apical microvilli, ONL thickness, and structural integrity of IPM.

41. The method of embodiment 40, wherein the marker is selected from BEST1, RPE65, EZRIN, pEZRIN, MCT1, CRALBP, F-actin, hCAR, an L-opsin, an M-opsin, an S-opsin, PNA, GFAP, Iba1, RDS/PRPH2, and RHO.

42. A method of identifying a subject in need of treatment for a bestrophinopathy, the method comprising:

performing in vivo retinal imaging on the subject to evaluate one or more of a longitudinal reflectivity profile (LRP), IS/OS to retinal pigment epithelium (RPE) distance in light-adapted and/or dark-adapted eyes, topological map, and formation of light-potentiated subretinal microdetachments,

identifying retinal changes indicative of Best-1 disease selected from one or more of abnormal POS-RPE apposition and microarchitecture of RPE-PR interface, elongation of both ROS & COS associated with increased ELM-RPE distance, accumulation of subretinal debris at RPE apical surface, or within subretinal space; compromised IPM and defective ELM; fluctuation of ONL thickness associated with reactive gliosis and cell migration; schistic changes inner/outer retina; formation of subretinal & intraretinal scars; RPE monolayer hypertrophy, occasional severe deformation of individual RPE cells associated with ONL & INL thickness fluctuations,

wherein a subject is identified as being in need of treatment for bestrophinopathy when one or more retinal changes indicative of Best1 disease is present.

43. The method of embodiment 42, wherein the performing in vivo retinal imaging comprises one or more of measurement of rod outer segments, cone outer segments, ONL thickness, and ELM-RPE distance.

44. The method of embodiment 42, wherein the performing in vivo retinal imaging comprises evaluation for reactive gliosis.

45. The method of any one of embodiments 42 to 44, wherein said retinal imaging is performed using an ultrahigh-resolution optical coherence tomography (OCT) to generate said LRP.

46. The method of any one of embodiments 42 to 45, wherein said retinal imaging comprises cSLO/SD-OCT, electrophysiology, or adaptation kinetics.

47. The method of any one of embodiments 41 to 46, further comprising treating the subject when one or more retinal changes indicative of Best1 disease is present.

48. The method according to embodiment 47, wherein the subject is treated using the method according to any one of embodiments 1 to 38.

49. The method according to any preceding claim, wherein the subject being treated is heterozygous for a BEST1 allele.

### REFERENCES

Aleman T S, Cideciyan A V, Aguirre G K, et al. Human CRB1-associated retinal degeneration: comparison with the rd8 Crb1-mutant mouse model. Invest Ophthalmol Vis Sci 2011; 52:6898-6910.
Beltran W A, Hammond P, Acland G M, Aguirre G D. A frameshift mutation in RPGR exon ORF15 causes photoreceptor degeneration and inner retina remodeling in a model of X-linked retinitis pigmentosa. Invest Ophthalmol Vis Sci 2006; 47(4):1669-1681.
Beltran W A, Cideciyan A V, Lewin A S, Iwabe S, Khanna H, et al. Gene therapy rescues X-linked photoreceptor blindness in dogs and paves the way for treating RPGR form of human retinitis pigmentosa. Proc Natl Acad Sci USA 2012; 109:2132-2137.
Beltran W A, Cideciyan A V, Guziewicz K E, Iwabe S, et al. Canine retina has a primate fovea-like bouquet of cone photoreceptors which is affected by inherited macular degenerations. PLoS One 2014; 9:e90390.
Boye S E, Huang W C, Roman A J, Sumaroka A, Boye S L, et al. Natural history of cone disease in the murine model of Leber congenital amaurosis due to CEP290 mutation: determining the timing and expectation of therapy. PLoS One. 2014; 9 (3):e92928.
Cideciyan A V, Jacobson S G, Aleman T S, Gu D, Pearce-Kelling S E, et al. In vivo dynamics of retinal injury and repair in the rhodopsin mutant dog model of human retinitis pigmentosa. Proc Natl Acad Sci USA 2005; 102:5233-5238.
Davidson A E, Millar I D, Urquhart J E, Burgess-Mullan R, et al. Missense mutations in a retinal pigment epithelium protein, bestrophin-1, cause retinitis pigmentosa. American journal of human genetics. 2009; 85:581-592.
Guziewicz K E, Zangerl B, Lindauer S J, Mullins R F, et al. Bestrophin gene mutations cause canine multifocal retinopathy: a novel animal model for Best disease. Invest Ophthalmol Vis Sci 2007; 48:1959-1967.
Guziewicz K E, Zangerl B, Komiromy A M, Iwabe S, et al. Recombinant AAV-mediated BEST1 transfer to the retinal pigment epithelium: analysis of serotype-dependent retinal effects. PLoS One 2013; 8:e75666.
Guziewicz K E, Sinha D, Gomez N M, Zorych K, et al. Bestrophinopathy: An RPE-photoreceptor interface disease. Prog Retin Eye Res 2017; 58:70-88.
Guziewicz K E, Cideciyan A V, Beltran W A, Komiromy A M, et al. BEST1 gene therapy corrects a diffuse retina-wide microdetachment modulated by light exposure. Proc Natl Acad Sci USA 2018; 115(12):E2839-E2848.
Huang W C, Wright A F, Roman A J, Cideciyan A V, Manson F D, et al. RPGR-associated retinal degeneration in human X-linked R P and a murine model. Invest Ophthalmol Vis Sci 2012; 53: 5594-608.
Zangerl B, Wickstrom K, Slavik J, Lindauer S J, Ahonen W, Schelling C, Lohi H, Guziewicz K E, Aguirre G D. Assessment of canine BEST1 variations identifies new mutations and establishes an independent bestrophinopathy model (cmr3). Mol Vis 2010; 16:2791-2804.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A method of treating a bestrophinopathy in a subject, comprising
administering to an eye of the subject a dose of a recombinant adeno-associated virus (rAAV) vector comprising a nucleic acid sequence encoding a human BEST1 protein,
wherein the subject has at least one mutant BEST1 allele, and
wherein the dose of the rAAV vector is:
a) administered at a concentration of about 1.0×10¹⁰ vector genomes (vg)/ml to about 1.0×10¹³ vg/ml; or
b) about 5.0×10⁸ vg per eye to about 5.0×10¹² vg per eye.

2. The method of claim 1, wherein the subject is a canine, mouse, rat, non-human primate, or human.

3. The method of claim 2, wherein the subject is a human.

4. The method of any one of claims 1 to 3, wherein the bestrophinopathy is Best Vitelliform Macular Dystrophy (BVMD), Autosomal dominant vitreoretinochoroidopathy (ADVIRC), Adult-onset vitelliform macular dystrophy (AVMD), retinitis pigmentosa (RP), or Microcornea, rod-cone dystrophy, and cataract.

5. The method of any of claims 1 to 4, wherein the rAAV vector is administered to the retina of the subject.

6. The method of any one of claims 1 to 4, wherein the rAAV vector is administered via subretinal, intravitreal, or suprachoroidal injection.

7. The method of claim 6, wherein the rAAV vector is administered via subretinal injection.

8. The method of any of claims 1 to 7, wherein the nucleic acid sequence expresses the human BEST1 protein in the retinal pigment epithelium (RPE) of the eye.

9. The method of any of claims 1 to 8, wherein the expression of the nucleic acid sequence encoding the BEST1 protein is under the control of a human VMD2 promoter (hVMD2).

10. The method of any of claims 1 to 9, wherein the dose of the rAAV vector is administered at a concentration of about 1.0×10¹⁰ vg/ml to about 3.0×10¹² vg/ml.

11. The method of claim 10, wherein the dose of rAAV vector is administered at a concentration of about 1.5×10¹⁰ vg/ml.

12. The method of any of claims 1 to 9, wherein the dose of rAAV vector is administered at a concentration of about 1.0×10¹¹ vg/ml to about 7.5×10¹¹ vg/ml.

13. The method of claim 12, wherein the dose of rAAV vector is administered at a concentration of about 3.0×10¹¹ vg/ml.

14. The method of claim 12, wherein the dose of rAAV vector is administered at a concentration of about 6.0×10¹¹ vg/ml.

15. The method of any of claims 1 to 9, wherein the dose of rAAV vector is administered at a concentration of about 7.5×10¹¹ vg/ml to about 1.0×10¹³ vg/ml.

16. The method of claim 15, wherein the dose of rAAV vector is administered at a concentration of about 3.5×10¹² vg/ml.

17. The method of any one of claims 1 to 16, wherein the dose of rAAV vector is administered in a volume of between about 50 ul and 500 ul.

18. The method of claim 17, wherein the dose of rAAV vector is administered in a volume of about 150 ul.

19. The method of claim 17, wherein the dose of rAAV vector is administered in a volume of about 300 ul.

20. The method of claim 17, wherein the dose of rAAV vector is administered in a volume of about 500 ul.

21. The method of any of claims 1 to 20, wherein the dose of rAAV vector administered is about 5.0×10⁸ vg per eye to about 1.5×10¹⁰ vg per eye.

22. The method of claim 21, wherein the dose of rAAV vector administered is about 7.5×10⁸ vg per eye.

23. The method of any of claims 1 to 20, wherein the dose of rAAV vector administered is about 1.0×10¹⁰ vg per eye to about 1.0×10¹¹ vg per eye.

24. The method of claim 23, wherein the dose of rAAV vector administered is about 4.5×10¹⁰ vg per eye.

25. The method of any of claims 1 to 20, wherein the dose of rAAV vector administered is about 1.0×10¹¹ vg per eye to about 5.0×10¹² vg per eye.

26. The method of claim 25, wherein the dose of rAAV vector administered is about 1.0×10¹² vg per eye.

27. The method of any one of claims 1 to 26, wherein the rAAV vector comprises an AAV2, AAV3, AAV4, AAV5, AAV6, AAV7, AAV8, AAV9, AAV10, AAV11, AAV12, LK01, LK02, LK03, AAV 4-1, AAV-2i8, Rh10, and/or Rh74 capsid, or a hybrid, chimera, or combination thereof.

28. The method of claim 27, wherein the rAAV vector comprises an AAV2 capsid, or a hybrid, chimera, or combination thereof.

29. The method of claim 28, wherein the rAAV vector comprises an AAV2 capsid.

30. The method of claim 29, wherein the rAAV vector is an AAV2-hVMD2-hBEST1 vector.

31. The method of any of claims 1 to 30, wherein the dose of rAAV is administered to each eye of the subject.

32. The method of any of claims 1 to 30, wherein the dose of rAAV is administered to both eyes of the subject.

33. The method of claim 1 to 32, wherein the method does not further comprise administration of a nucleic acid composition that suppresses the expression or activity of the at least one mutant BEST1 allele.

34. The method of any of claims 1 to 33, wherein treatment of the bestrophinopathy is evaluated comprising:
performing in vivo retinal imaging to evaluate one or more of a longitudinal reflectivity profile (LRP), IS/OS to retinal pigment epithelium (RPE) distance in light-adapted and/or dark-adapted eyes, electrophysiology, dark-adapted kinetic perimetry and formation of light-potentiated subretinal microdetachments,
wherein treatment efficacy is indicated by one or more of a rescue of retinal microarchitecture through restoration of RPE apical microvilli structure, and a reestablishment of proper apposition between RPE cells and photoreceptor (PR) outer segments (cytoarchitecture of RPE-PR interface).

35. The method of claim 34, wherein the performing in vivo retinal imaging comprises one or more of fundus examination, cSLO/SD-OCT, measurement of rod outer segments, cone outer segments, ONL thickness, and ELM-RPE distance.

36. The method of claim 34, wherein the performing in vivo retinal imaging comprises evaluation for reactive gliosis.

37. The method of claim 34, further comprising evaluation for Muller glial trunks/projections penetrating ONL layer with astrogliosis.

38. The method of any one of claims 34 to 37, wherein said retinal imaging is performed using an ultrahigh-resolution optical coherence tomography (OCT) to generate said LRP.

39. The method of any one of claims 34 to 38, further comprising comparing a measurement of a selected parameter to a measurement in a normal control, mutant disease control, pre-treatment control, earlier timepoint control, an untreated contralateral eye, or a retinal region outside of a treatment bleb.

40. The method of any one of claim 34 to 39, further comprising obtaining a retina sample from the treated subject and
a) labeling the sample with at least one RPE- and/or photoreceptor-specific marker;
b) obtaining high-resolution confocal or wide-field fluorescence microscope with Differential Interference Contrast (DIC) option images of the RPE-PR interdigitation zone; and
c) assessing one or more of length of RPE apical microvilli, structure of apical microvilli, ONL thickness, Muller Glial trunks/projections penetrating ONL layer, and structural integrity of IPM.

41. The method of claim 40, wherein the marker is selected from BEST1, RPE65, EZRIN, pEZRIN, MCT1, CRALBP, F-actin, hCAR, an L-opsin, an M-opsin, an S-opsin, PNA, GFAP, Iba1, RDS/PRPH2, and RHO.

42. A method of identifying a subject in need of treatment for a bestrophinopathy, the method comprising:
performing in vivo retinal imaging on the subject to evaluate one or more of a longitudinal reflectivity profile (LRP), IS/OS to retinal pigment epithelium (RPE) distance in light-adapted and/or dark-adapted eyes, topological map, and formation of light-potentiated subretinal microdetachments,
identifying retinal changes indicative of Best-1 disease selected from one or more of abnormal POS-RPE apposition and microarchitecture of RPE-PR interface, elongation of both ROS & COS associated with increased ELM-RPE distance, accumulation of subretinal debris at RPE apical surface, or within subretinal space; compromised IPM and defective ELM; fluctuation of ONL thickness associated with reactive gliosis and cell migration; schistic changes inner/outer retina; formation of subretinal & intraretinal scars; RPE monolayer hypertrophy, occasional severe deformation of individual RPE cells associated with ONL & INL thickness fluctuations,
wherein a subject is identified as being in need of treatment for bestrophinopathy when one or more retinal changes indicative of Best1 disease is present.

43. The method of claim 42, wherein the performing in vivo retinal imaging comprises one or more of measurement of rod outer segments, cone outer segments, ONL thickness, and ELM-RPE distance.

44. The method of claim 42, wherein the performing in vivo retinal imaging comprises evaluation for reactive gliosis.

45. The method of claim 42, wherein the performing in vivo retinal imaging comprises cSLO/SD-OCT.

46. The method of any one of claims 42 to 45, wherein said retinal imaging is performed using an ultrahigh-resolution optical coherence tomography (OCT) to generate said LRP.

47. The method of any one of claims 42 to 46, wherein said retinal imaging comprises electrophysiology, or adaptation kinetics.

48. The method of any one of claims 41 to 47, further comprising treating the subject when one or more retinal changes indicative of Best1 disease is present.

49. The method according to claim 48, wherein the subject is treated using the method according to any one of claims 1 to 38.

50. The method according to any of claims 1 to 41, wherein the subject being treated is heterozygous for a BEST1 allele.
